(19) United States
(12) Patent Application Publication
(10) Pub. No.: US 2008/0167641 A1
(43) Pub. Date: Jul. 10, 2008

(54) Medical Device Adapted To Detect Disengagement Of A Transcutaneous Device

(75) Inventors: Steffen Hansen, Hillerod (DK); Ole Christian Nielsen, Hillerod (DK); Henrik Bengtsson, Frederiksberg (DK); Jens Peter Jensen, Jyllinge (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/913,689

(22) PCT Filed: May 15, 2006
(86) PCT No.: PCT/EP06/62301

§ 371 Date: Dec. 12, 2007

(30) Foreign Application Priority Data
May 13, 2005 (DK) 2005 00703

(51) Int. Cl. A61M 5/14

(52) U.S. Cl. 604/891.1; 604/502

(57) ABSTRACT

The present invention provides a medical device comprising a transcutaneous device. The medical device further comprises a controller for detecting a first condition representative of the transcutaneous device being arranged in a subcutaneous first position, and for detecting a second condition representative of the transcutaneous device being arranged in a non-subcutaneous second position, wherein the controller is adapted for actuating an alarm when a condition representative of the transcutaneous device being arranged in a non-sub-cutaneous position is detected.

The present invention relates to a medical device comprising a transcutaneous device adapted to be arranged subcutaneously in a subject. In a specific aspect, the invention relates to such a device adapted to detect a condition which may lead to failure in the controlled delivery of an amount of drug to a subject.

### BACKGROUND OF THE INVENTION

In the disclosure of the present invention reference is mostly made to the treatment of diabetes by injection or infusion of insulin, however, this is only an exemplary use of the present invention.

Portable drug delivery devices for delivering a drug to a patient are well known and generally comprise a reservoir adapted to contain a liquid drug and having an outlet in fluid communication with a transcutaneous access device such as a hollow infusion needle or a cannula, as well as expelling means for expelling a drug out of the reservoir and through the skin of the subject via the access device. Such drug delivery devices are often termed infusion pumps.

Basically, infusion pumps can be divided into two classes. The first class comprises infusion pumps which are relatively expensive pumps, e.g. as known from U.S. Pat. No. 5,647,853, intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals.

Addressing the above problem, several attempts have been made to provide a second class of drug infusion devices that are low in cost and convenient to use. Some of these devices are intended to be partially or entirely disposable and may provide many of the advantages associated with an infusion pump without the attendant cost and inconveniencies, e.g. the pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump (also known as a bleeding hole pump)), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference.

Irrespective of the type of pump technology used, it is desirable to monitor proper functioning of an actuated drug delivery device or system, and thus to provide means for detecting different operational conditions of the system, such as an occlusion condition downstream of a pump, e.g. full or partial occlusion of a transcutaneous access device. As the outlet conduit leading from the pump outlet to the distal outlet opening of a transcutaneous access device is relatively stiff, a given pressure rise in the outlet conduit during pump actuation can normally be taken as an indication for an occlusion condition and thus be utilized to detect the latter. For example, US 2004/0127844 discloses a delivery device comprising pressure sensors being actuated by a resilient diaphragm arranged in flow communication with in the outlet conduit. U.S. Pat. No. 6,555,986 describes a method and apparatus for automatically detecting an occlusion or drive system failure in a medication infusion system is provided. The electrical current to an infusion pump is measured and compared against a baseline average current. If the current exceeds a threshold amount, an alarm is triggered. Alternatively, pump motor encoder pulses are measured during a pump cycle. U.S. Pat. No. 5,647,853 describes an occlusion detector provided in a medication infusion pump and comprising a force sensor for reading and comparing the pressures applied to the medication. U.S. Pat. No. 4,544,369 describes occlusion detection for small infusion pump. WO 90/07942 discloses a method and apparatus for continuous monitoring of the operation of a drug delivery system. The above cited documents are hereby incorporated by reference.

Before turning to the disclosure of the present invention, a different type of skin-mountable device will be described. Although drug infusion pumps, either disposable or durable, may provide convenience of use and improved treatment control, it has long been an object to provide a drug infusion system for the treatment of e.g. diabetes which would rely on closed loop control, i.e. being more or less fully automatic, such a system being based on the measurement of a value indicative of the condition treated, e.g. the blood glucose level in case of insulin treatment of diabetes.

A given monitor system for measuring the concentration of a given substance may be based on invasive or non-invasive measuring principles. An example of the latter would be a non-invasive glucose monitor arranged on the skin surface of a patient and using near-IR spectroscopy. The sensor may be placed subcutaneously being connected to external equipment by wiring or the substance (fluid) to be analysed may be transported to an external sensor element, both arrangements requiring the placement of a subcutaneous component, the present invention addressing both arrangements. However, for simplicity the term “sensor” is used in the following for both types of sensor elements.

Turning to the sensor elements per se, relatively small and flexible electrochemical sensors have been developed for subcutaneous placement of sensor electrodes in direct contact with patient blood or other extra-cellular fluid (see for example U.S. Pat. No. 5,482,473), wherein such sensors can be used to obtain periodic or continuous readings over a period of time. Insertion devices for this type of sensors are described in, among others, U.S. Pat. Nos. 5,390,671, 5,391,950, 5,568,806 and 5,954,643 which hereby are incorporated by reference.

Although the above-described detection systems are capable of identifying certain conditions in which a drug is not delivered to a patient in accordance with given settings, there is a need for improved methods and apparatus for detecting additional conditions which may lead to failure in the controlled delivery of an amount of drug to a subject in accordance with given settings.

It is a further object to provide methods and apparatus which can be applied to and used in combination with a broad range of drug delivery technologies.

It is a further object to provide an actuator system which allows for detection of different operational conditions of the system, thereby ideally providing a system which can be actuated and controlled in a safe and efficient manner.

It is a further object to provide an actuator system which can be used in combination with a pump assembly arranged in a portable drug delivery device, system or a component therefore, thereby providing controlled infusion of a drug to a subject. It is a further object to provide an actuator system which can be used in combination with a pump such as a membrane pump. It is a further object of the invention to provide an actuator, or component thereof, which can be provided and applied in a cost-effective manner.

Based on the principles for detecting conditions which may lead to failure in the controlled delivery of an amount of drug to a subject, it is a further object of the present invention to provide solutions adapted for also detecting failure with respect to the correct placement of a transcutaneous device in general, e.g. a sensor.

### DISCLOSURE OF THE INVENTION

In the disclosure of the present invention, embodiments and aspects will be described which will address one or more of the above objects or which will address objects apparent from the below disclosure as well as from the description of exemplary embodiments.

According to a first aspect of the invention, a drug delivery device is provided, comprising a transcutaneous access device adapted to be arranged subcutaneously in a subject, a reservoir adapted to contain a fluid drug, and an expelling assembly adapted for cooperation with the reservoir to expel fluid drug out of the reservoir and through the transcutaneous access device. The device further comprises a controller for detecting a first condition representative of the transcutaneous access device being arranged in a first subcutaneous position, and for detecting a second condition representative of the transcutaneous access device being arranged in a second non-subcutaneous position, wherein the controller is adapted for performing an action corresponding to the detection of the second condition. The action may be in the form of a “positive” action, e.g. actuating an audible, visual or tactile alarm or initiating a modified actuation pattern after detection of the second condition, or it may be a “silent” action, e.g. transmitting a given signal which can then be used by other components associated with the device. The detection of the first condition may be “implicit”, i.e. it merely represents a “no event”. For example, a detected value is compared with a stored value and dependent upon the detected value being above or below the stored value, the processor will perform an action or no action.

When in the context of the present disclosure of the invention and in the claims it is defined that the drug delivery device comprises a transcutaneous access device, this definition also covers the situation in which the device does not comprise a transcutaneous access device but is adapted to be connected to and used in combination with a transcutaneous access device, e.g. an infusion set. Correspondingly, a drug delivery device is provided, comprising or being adapted to be connected to a transcutaneous device, the transcutaneous device being adapted to be arranged subcutaneously in a subject.

In a further aspect of the invention a drug delivery device is provided, comprising a transcutaneous access device adapted to be arranged subcutaneously in a subject, a reservoir adapted to contain a fluid drug, and an expelling assembly adapted for cooperation with the reservoir to expel fluid drug out of the reservoir and through the transcutaneous access device. The device further comprises a controller for detecting a condition representative of the transcutaneous access device being arranged in a non-subcutaneous position, and for performing an action in response thereto.

Correspondingly, in an exemplary embodiment the controller is adapted for actuating an alarm when a condition representative of the transcutaneous access device being arranged in a non-subcutaneous position is detected. The delivery device may comprise indication means adapted to indicate to a user that the transcutaneous access device is arranged in a non-subcutaneous position, e.g. a display indicating “check placement of cannula” or a specific audible alarm pattern, this allowing a user to be directly informed as to the reason for the alarm condition.

A drug delivery device may be a unitary device or it may be in the form of a system, thus a drug delivery device in accordance with aspects of the present invention may comprise a delivery unit in which the reservoir and expelling assembly are arranged, and a remote unit comprising indication means adapted to indicate to a user that the transcutaneous access device is arranged in a non-subcutaneous position. The remote unit may be a wireless controller allowing the user to access and control the delivery unit via the remote unit.

In the context of the present application and as used in the specification and claims, the term controller covers any combination of electronic circuitry and associated components, e.g. sensors, suitable for providing the specified functionality, e.g. sensing properties, processing data and controlling memory as well as all connected input and output devices. The controller may comprise one or more processors or CPUs which may be supplemented by additional devices for support or control functions. For example, the detection means, a transmitter, or a receiver may be fully or partly integrated with the controller, or may be provided by individual units. Each of the components making up the controller circuitry may be special purpose or general purpose devices. The detection means may comprise a “sensor” per se, e.g. in the form of an electrical contact, a pressure sensor or an optical or magnetic sensor capable of being influenced by a given property.

The controller may be adapted to detect a first condition associated with the subcutaneous delivery of drug, and detect a second condition associated with the non-subcutaneous delivery of drug, e.g. expelling fluid against a first higher resistance respectively expelling fluid against a second lower resistance. In this way a situation can be detected in which a normal amount of drug is delivered, however, not at the desired location.

A second condition indicative of non-subcutaneous delivery of drug may arise although the transcutaneous access device is actually arranged subcutaneously. For example, in case of a leakage between the expelling assembly and the transcutaneous access device or between the reservoir and the transcutaneous access device, or in case of a reservoir running empty, the controller may detect values indicative of a lower flow resistance and thus indicate or activate an alarm. Although the alarm would thus not indicate non-subcutaneous delivery of drug, it would still provide very useful information indicative of a malfunction of the delivery device and thus inadequate delivery of drug.

To provide the user with more specific information as to the possible reason for an alarm condition, the controller may be adapted to distinguish between different conditions associated with a low flow resistance. For example, as the flow resistance in the transcutaneous access device per se may represent a non-neglectable flow resistance, a high drop in flow resistance during expelling of drug may be indicative of a leak upstream of the transcutaneous access device, e.g. an external flexible tube connecting an infusion set with a delivery device may have disengaged. In a further example, the controller may be provided with information as to the amount of drug left in the reservoir such that a low level of drug in the reservoir would not trigger an alarm indicative of non-subcutaneous delivery of drug due to low pressure (but may indeed trigger an indication that the reservoir is close to empty). In a yet further example, the delivery device may be provided with a flow sensor actually measuring the amount of expelled drug (e.g. based on thermo-dilution), this allowing the controller to detect the second condition when fluid drug is expelled from the transcutaneous access device at substantially the same rate as in the first condition.

To properly detect the second condition representative of the transcutaneous access device being arranged in a second non-subcutaneous position, the controller may be adapted to evaluate detected values and implement a “strategy” to avoid false positive determinations of the second condition. For example, when a user moves with the transcutaneous access device arranged subcutaneously, the flow resistance in the subcutaneously tissue may vary “naturally”. Correspondingly, the controller may be adapted to evaluate a number of “second condition values” within a given time range before performing an action corresponding to the detection of the second condition.

The controller may be adapted to detect a first condition associated with the pressure in the transcutaneous access device during subcutaneous delivery of drug and to detect a second condition associated with the pressure in the transcutaneous access device during non-subcutaneous delivery of drug. Alternatively, the controller may be adapted to detect a first condition associated with a first pressure in the transcutaneous access device during delivery of drug, and to detect a second condition associated with a lower pressure in the transcutaneous access device during delivery of drug.

In an exemplary embodiment the controller comprises a pressure sensor in fluid communication with the transcutaneous access device, and may comprise information representing a first pressure range or pressure pattern associated with the first condition, and a second pressure range or pressure pattern associated with the second condition.

In a further exemplary embodiment the controller comprises a current sensor for sensing current supplied to the expelling assembly, and may comprise information representing a first current range or current pattern associated with the first condition, and a second current range or current pattern associated with the second condition.

In a yet further exemplary embodiment the controller comprises a position sensor for sensing a position of a structure moved during operation of the expelling assembly. Correspondingly, the expelling assembly may comprise actuating means moveable between first and second positions, the controller comprising detection means for detecting a lapsed time or time pattern when the actuating means is moved between the first and second positions in a given direction. The controller may comprise information representing a first lapsed time range or time pattern associated with the first condition, and a second lapsed time range or time pattern associated with the second condition.

For example, a first high pressure (or current or time) range may be associated with a peak pressure rise during subcutaneous delivery of drug due to flow resistance in the subcutaneous tissue, and a second lower pressure range may be associated with a lower peak pressure rise during non-subcutaneous delivery of drug due to the lower flow resistance when pumping drug out of the free end of the transcutaneous access device. In respect of a detected pattern, a small basal rate infusion may be associated with a peak pressure rise due to flow resistance in the flow conduit and transcutaneous access device and a slow decrease in pressure as the drug dissipates into the subcutaneous tissue, or a small basal rate infusion may be associated with substantially the same peak pressure rise due to flow resistance in the flow conduit and transcutaneous access device but a faster decrease in pressure in case the drug escapes from the free end of the transcutaneous access device.

The different value ranges may be predefined, selectable or they may be dynamically influenced by actuation history over a short or long period of time. The range(s) may be closed, open or open-ended, e.g. a time range may be “closed” (e.g. 50-100 ms) or “open” (e.g. >50 ms or <100 ms). For example, upon initial use of a given actuated system in which it is known that a transcutaneous access device is properly arranged subcutaneously, the system may be actuated a number of times (e.g. when priming a pump), and the values detected (e.g. pressure, current or time) during these actuations be used to determine a value which is unique for the actual system, which value may then be used to calculate one or more defined ranges to be used for the subsequent determination of different conditions for the system. As a safety feature, the actuator system may be provided with preset values or ranges within which the dynamically determined ranges should fall, this to prevent that a dynamic range is determined for a defective system.

Correspondingly, in an exemplary embodiment the invention provides a drug delivery device as set out above, wherein the controller is adapted to operate the expelling assembly in accordance with a first mode, detect a value for a property associated with operation of the expelling assembly (e.g. a pressure, current or time value), and provide a first value range when the expelling assembly is operated during the first mode, the first value range being indicative of subcutaneous delivery of drug when the transcutaneous access device is arranged subcutaneously. The controller is further adapted to on the basis of the first value range to provide a second value range, the second value range being indicative of non-subcutaneous delivery of drug, operate the expelling assembly in accordance with a second mode, detect a value for the property when the expelling assembly is operated during the second mode, and perform an action when a detected value is within the second value range. The second mode may e.g. be infusion in accordance with a basal rate profile or a bolus injection. In other embodiments the first and second modes may be chosen in accordance with the desired application.

In the above-described exemplary embodiments, the controller has been adapted to detect a first condition associated with the subcutaneous delivery of drug, and detect a second condition associated with the non-subcutaneous delivery of drug, however, the controller may be adapted to detect a first condition associated with actual subcutaneous placement of the transcutaneous access device, respectively a second condition associated with non-subcutaneous placement of the of the transcutaneous access device. For example, the transcutaneous access device may comprise a sensor influenced by a property associated with subcutaneous placement of a distal portion thereof, e.g. temperature, electrical conductance or pH.

In an exemplary embodiment a drug delivery device as described above is provided, further comprising a mounting surface adapted for application to a skin surface of the subject, wherein the transcutaneous access device comprises a distal end adapted to be inserted through the skin of the subject, the distal end being moveable between an initial position in which the distal end is retracted relative to the mounting surface, and an extended position in which the distal end projects relative to the mounting surface.

In an alternative exemplary embodiment a drug delivery device as described above is provided, further comprising a housing in which the reservoir and expelling assembly is at least partially arranged, the transcutaneous access device being arranged outside the housing and connected thereto by means of a flexible tube.

The expelling assembly may be of any suitable type, e.g. as described above in the introductory portion, the actual type of expelling assembly determining what kind of property can be detected. For example, an electrically driven piston pump may allow detection of supplied current, a reciprocating actuator (e.g. a coil or SMA actuator) may allow time values to be detected, whereas a gas generator may need an additional pressure sensor which, indeed, may also be used in combination with an electric motor or actuator. Further, more than one property may be detected to determine a non-subcutaneous position of a transcutaneous access device.

The reservoir may be any suitable structure adapted to hold an amount of a fluid drug, e.g. a hard reservoir, a flexible reservoir, a distensible or elastic reservoir. The reservoir may e.g. be prefilled, user fillable or in the form of a replaceable cartridge which again may be prefilled or fillable.

The transcutaneous access device may e.g. be in the form of a hollow steel needle, a soft cannula (e.g. made from or comprising PCTFE or from any other suitable polymeric material) in combination with an external or internal introduction needle, or a micro-needle array.

In a further embodiment the present invention provides an occlusion detector for use with a medication infusion pump comprising a transcutaneous access device adapted to be arranged subcutaneously in a subject, a reservoir adapted to contain a fluid drug, and an expelling assembly adapted for cooperation with the reservoir to expel fluid drug out of the reservoir and through the transcutaneous access device. The occlusion detector comprises sensor means for providing output signals representative of the pressure in the transcutaneous access device, control circuit means connected to the sensor means for receiving the output signals, the control circuit means including comparator means for comparing the difference between a first output signal corresponding to a first pressure during a first operation of the expelling assembly and associated with the transcutaneous access device being arranged in a first subcutaneous position, and a second output signal corresponding to a second lower pressure during a second operation of the expelling assembly and associated with the transcutaneous access device being arranged in a second non-subcutaneous position, and alarm means activated by said control circuit means when the difference between said first and second output signals is more than a predetermined value indicative of a non-subcutaneous position of the transcutaneous access device.

In a further aspect a method for operating a drug delivery device comprising a transcutaneous access device is provided, comprising the steps of (i) arranging the transcutaneous access device subcutaneously in a subject, (ii) detecting a condition influenced by the transcutaneous access device being arranged in a subcutaneous or non-subcutaneous position, and (iii) generating an alarm when a condition indicative of the transcutaneous access device being arranged in a non-subcutaneous position is detected.

In a further aspect a method for operating a drug delivery device comprising a reservoir, an expelling assembly and a transcutaneous access device is provided, the method comprising the steps of (i) operating the expelling assembly to expel fluid drug out of the reservoir through the transcutaneous access device, (ii) detecting a condition associated with the pressure in the transcutaneous access device during delivery of drug, and (iii) actuating an alarm when the detected condition is associated with a pressure below a defined value. The condition associated with the pressure in the transcutaneous access device may e.g. be pressure, current or time as discussed above.

In a further aspect a method for operating a drug delivery device comprising a reservoir, an expelling assembly and a transcutaneous access device is provided, the method comprising the steps of (i) operating the expelling assembly to expel fluid drug out of the reservoir through the transcutaneous access device, (ii) detecting a property associated with operation of the expelling assembly, and (iii) determining on basis of the detected property whether drug is expelled subcutaneously in a subject or non-subcutaneously.

In a yet further aspect a method for operating a drug delivery device comprising a reservoir, an expelling assembly and a transcutaneous access device is provided, the method comprising the steps of (i) arranging the transcutaneous access device subcutaneously in a subject, (ii) operating the expelling assembly to expel fluid drug out of the reservoir and through the transcutaneous access device, (iii) detecting a property associated with operation of the expelling assembly, and (iv) determining on basis of the detected property whether drug is expelled subcutaneously in the subject or non-subcutaneously.

In a further aspect a method for operating a drug delivery device comprising a reservoir, an expelling assembly and a transcutaneous access device is provided, the method comprising the steps of (i) arranging the transcutaneous access device subcutaneously in a subject, (ii) operating the expelling assembly to expel fluid drug out of the reservoir and through the transcutaneous access device, (iii) detecting a property associated with operation of the expelling assembly, (iv) determining a first value or range for the property with the transcutaneous access device arranged subcutaneously, (v) determining on the basis of the first value or range a second range indicative of the transcutaneous access device being arranged non-subcutaneously, and (vi) performing an action, e.g. activating an alarm, when a value or pattern for the property within the second range is detected during operation of the expelling assembly. For example, during operation of the expelling assembly with the transcutaneous access device arranged subcutaneously, a value of e.g. 100 ms, or a range of e.g. 90-110 ms may be determined, on which a second range of e.g. <50 ms or <45 ms may be determined, the latter ranges then representing values indicative of a non-subcutaneously arrangement of the transcutaneous access device.

In the above-described methods the property may be representative of the pressure in the transcutaneous access device during operation of the expelling assembly. The methods may comprise the additional step of indicating to a user that the transcutaneous access device is arranged in a non-subcutaneous position. The steps of the methods of the present invention may be performed in any suitable order.

The invention also provides a computer program product for carrying out a method as disclosed above when the computer program product is executed in a drug delivery device comprising a transcutaneous access device adapted to be arranged subcutaneously in a subject, a reservoir adapted to contain a fluid drug, an expelling assembly adapted for cooperation with the reservoir to expel fluid drug out of the reservoir and through the transcutaneous access device, and a controller (comprising e.g. a computer or a microprocessor with a flash memory) adapted to detect a condition influenced by the subcutaneous or non-subcutaneous position of the transcutaneous access device.

In the above disclosure of aspects of the present invention reference has been made to a drug delivery device, however, in a further aspect the principles of the present invention may be used for other types of medical devices comprising a transcutaneous device. Thus, in a further aspect, the present invention provides a medical device comprising a transcutaneous device adapted to be arranged subcutaneously in a subject, a controller for detecting a first condition representative of the transcutaneous device being arranged in a subcutaneous first position, and for detecting a second condition representative of the transcutaneous device being arranged in a non-subcutaneous second position, wherein the controller is adapted for performing an action corresponding to the detection of the second condition. The transcutaneous device may be in the form of e.g. a sensor and the sensed property may e.g. be a chemical, biological, fluid or temperature condition. The controller may be adapted to operate the device in accordance with a first mode, detect a value for a property associated with the first mode and provide a first value range being indicative of a first condition, on the basis of the first value range provide a second value range, the second value range being indicative of a second condition, operate the device in accordance with a second mode and detect a value for the property when the device is operated during the second mode, and perform an action when a detected value is within the second value range. The first mode may be initial priming or set-up measuring of a sensor device, and the first condition is associated with the sensor device being arranged subcutaneously, whereas the second condition is non-subcutaneous placement of the sensor device.

In a yet further aspect of the present invention, a medical device is provided, comprising a mounting surface adapted for application towards a skin surface of a subject, a flexible transcutaneous device comprising a distal portion adapted to be arranged subcutaneously in the subject through a point of insertion, wherein the distal portion comprises a visual marking, and whereby the distal portion is readily identifiable by the naked eye of a user should the transcutaneous device disengage from its intended subcutaneous position. The visual marking may be in the form of a colour marking arranged at the distal end of the transcutaneous device, or the visual marking may be arranged along the length of the distal portion of the transcutaneous device. In a situation of use in which the medical device is applied towards the skin surface of the subject, the medical device may be adapted to allow a point of insertion to be observed directly by the user, this allowing the user to detect a condition in which the transcutaneous device has disengaged from its intended subcutaneous position. The transcutaneous device may be moveable from a retracted position to an extended position relative to the mounting surface.

In a yet further aspect of the present invention, a medical device is provided, comprising a mounting surface adapted for application towards a skin surface of a subject, a first electrode and a second electrode. The device further comprises means for detecting a capacitance between the first and second electrodes, wherein the first electrode is in the form of a transcutaneous device comprising a distal portion adapted to be arranged subcutaneously in the subject, the transcutaneous device being conductive in a situation of use, and the second electrode is in the form of a skin-mountable electrode. The transcutaneous device may be conductive or it may be hollow and substantially non-conductive, the fluid-filled transcutaneous device providing a conductive transcutaneous device in a situation of use.

In an exemplary embodiment the medical device further comprises means for applying an AC voltage between the two electrodes, a controller for detecting a first condition determined by a first range of capacitance values representative of the transcutaneous access device being arranged in a subcutaneous first position, and for detecting a second condition determined by a second range of capacitance values representative of the transcutaneous access device being arranged in a non-subcutaneous second position, wherein the controller is adapted for performing an action corresponding to the detection of the second condition. The controller may be adapted to detect a capacitance value when the medical device is operated during a first mode, on the basis of the detected capacitance value provide a first capacitance value range, the first capacitance value range being indicative of a first condition of placement of the transcutaneous device, on the basis of the first value range provide a second capacitance value range, the second value range being indicative of a second condition of placement of the transcutaneous device, operate the medical device in accordance with a second mode, detect a capacitance value when the medical device is operated during the second mode, and perform an action when a detected capacitance value is within the second value range. The first mode may be an initial mode associated with the transcutaneous access device being arranged subcutaneously in the subject, the first condition being associated with the transcutaneous device being arranged subcutaneously with the second electrode in contact with the skin surface, and the second condition being associated with the transcutaneous device being arranged non-subcutaneously with second electrode in contact with the skin surface.

For the above-described embodiments the second electrode may associated with the mounting surface or it may be attached to the medical device in other ways, e.g. it may be a separate patch electrode connected to a durable-type drug infusion pump, the mounting surface being provided by an infusion set carrying a cannula.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals (including peptides, proteins, and hormones), biologically derived or active agents, hormonal and gene based agents, nutritional formulas and other substances in both solid (dispensed) and liquid form. In the description of the exemplary embodiments reference will be made to the use of insulin. Correspondingly, the term “subcutaneous” infusion is meant to encompass any method of parenteral delivery to a subject.

### BRIEF DESCRIPTION OF THE DRAWINGS

In the following the invention will be further described with references to the drawings, wherein

FIG. 1 shows an exploded view of an embodiment of an actuator in combination with a pump,

FIGS. 2A-2C show schematic cross-sectional views through a pump and actuator assembly in different stages of actuation,

FIGS. 3A and 3B show schematic cross-sectional views through a part of a further pump and actuator assembly,

FIG. 4 shows a cross-sectional view through piston rod mounted in a pump,

FIG. 5 shows an exploded view of a further embodiment of an actuator,

FIG. 6 shows the actuator of FIG. 5 in an assembled state,

FIG. 7 shows a cross-sectional view of the actuator of FIG. 5,

FIG. 8 shows the actuator of FIG. 5 in an assembled state with a flex print mounted,

FIGS. 9A-9C show cross-sectional views through the actuator assembly of FIG. 5 in different stages of actuation,

FIG. 10 shows the patch unit of FIG. 5 in greater detail,

FIG. 11 shows the patch unit of FIG. 7 in an actuated state,

FIG. 12 shows a patch unit with a pump unit partly attached,

FIG. 13 shows the pump unit of FIG. 9 fully attached to the patch unit,

FIG. 14 shows in an exploded view a schematic representation of a transcutaneous device unit,

FIGS. 15A-15D show in different actuation states a mechanism for insertion of a cannula,

FIG. 16 shows in an exploded view a pump unit,

FIG. 17 shows a diagram representing controller evaluation of actuator derived information,

FIG. 18 shows T-out in milliseconds (ms) during actuation of a pump,

FIG. 19 shows the pressure (mbar) during actuation of a pump,

FIGS. 20 and 21 show two further embodiments of a drug delivery device,

FIGS. 22A-22H show a cannula in combination with different means for detection of cannula position, and

FIGS. 23A and 23B show a marked cannula in different positions.

In the figures like reference numerals are used to mainly denote like or similar structures.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as their relative dimensions are intended to serve illustrative purposes only.

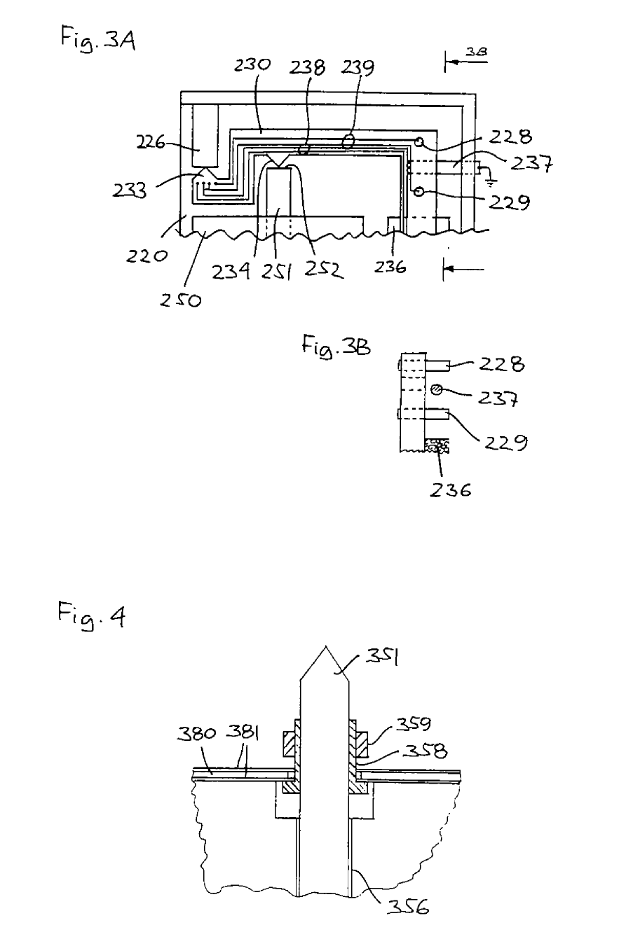

More specifically, a pump actuator 1 comprises an upper housing member 10 and a lower housing member 20, both comprising a distal main portion 11, 21 and a there from extending proximal arm portion 12, 22. On an upper surface of the lower main portion a pair of opposed walls 23, 24 are arranged and at the proximal end of the lower arm a post member 25 and a knife-edge member 26 are arranged perpendicularly to the general plane of the lower arm. In an assembled state the two main portions form a housing in which a pair of magnets 40, 41 is arranged on the opposed upper and lower inner surfaces of the main portions. The pump actuator further comprises a lever 30 having a proximal end 31 comprising first and second longitudinally offset and opposed joint structures in the form of a groove 33 and a knife-edge 34 arranged perpendicular to a longitudinal axis of the lever, and a distal end 32 with a pair of gripping arms 35 for holding a coil member 36 wound from a conductor. A membrane pump is arranged in a pump housing 50 having a bore in which an actuation/piston rod 51 is arranged, the rod serving to actuate the pump membrane of the membrane pump (see below for a more detailed description of a membrane pump). The outer free end of the rod is configured as a substantially planar surface 52. In an assembled state the lever is arranged inside the housing with the coil positioned between the two magnets, and the housing is attached to the pump housing with the knife-edge of the knife-edge member 26 nested in the lever groove 33 and the knife-edge of the lever is positioned on the planar rod end surface, this arrangement providing first and second pivoting joints. As the actuating rod is biased outwardly by the elastic pump membrane the lever is held in place by the two joints and the housing in combination, the lever only being allowed to pivot relative to the first joint (see also below). Due to this arrangement a gearing of the force provided from the coil-magnet actuator to the actuation rod is realized, the gearing being determined by the distance between the two pivoting joints (i.e. a first actuator arm) and the distance between the first/proximal pivoting joint and the “effective” position of the coil on the lever (i.e. a second actuator arm). By the term “effective”, the issue is addressed that the force generated by the coil actuator may vary as a function of the rotational position of the lever, this being due to the fact that the coil is moved between stationary magnets, which may result in a varying magnetic field for the coil as it is moved. The actuator further comprises a pair of contact members 28, 29 adapted to cooperate with a contact rod 37 mounted in the housing and which will be described with reference to FIG. 3A.

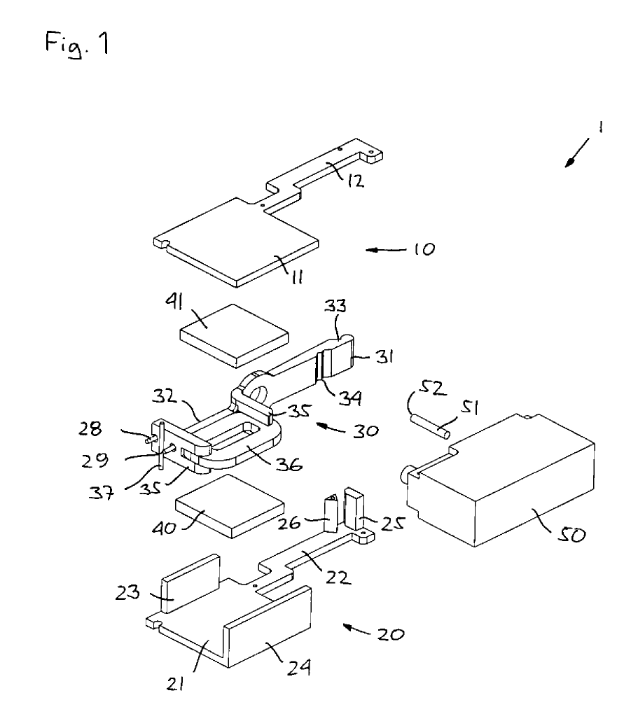

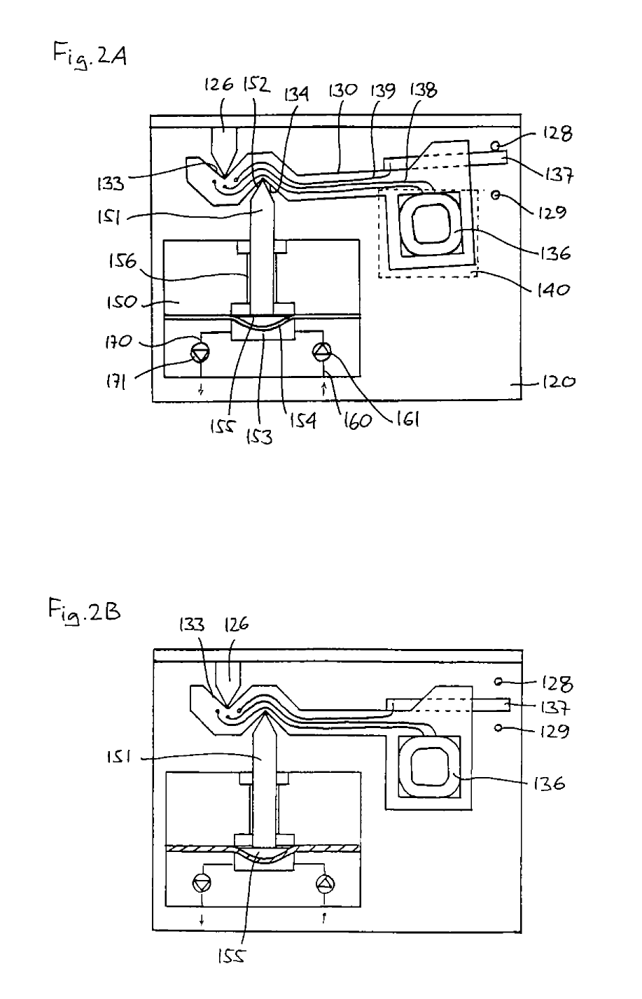

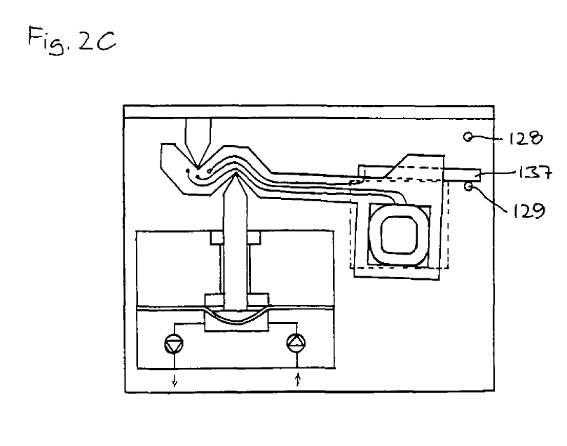

FIGS. 2A-2C show schematic cross-sectional views through a pump and actuator assembly of the type shown in FIG. 1, the sections corresponding to a plane above the lever. Corresponding to the FIG. 1 embodiment, the assembly comprises a housing 120 for accommodating the actuator lever 130, a pair of magnets 140 as well as a pump assembly 150, the housing comprising a knife-edge member 126. The pump assembly may be of the type disclosed in FIGS. 11-16. The actuator lever comprises first and second grooves 133, 134, a coil 136 and a contact rod 137 adapted to engage first and second contact members 128, 129 arranged on the housing. The lever further comprises a pair of conductors 138 for energizing the coil as well as a conductor 139 for the contact rod. In the shown embodiment the conductors are shown with terminal contact points, however, advantageously the three conductors are formed on a flex-print attached to the lever and connected to a structure of the device in which the actuator is mounted, the connection between the moving lever and the other structure being provided by a film hinge formed by the flex-print. The pump comprises a pump chamber 153, in which an elastic pump membrane 154 is arranged, and a bore 156 for slidingly receive and support a piston rod 151 with a convex piston head 155 engaging the pump membrane. The pump membrane is in all positions in a stretched state, the membrane thereby exerting a biasing force on the piston rod which is used to hold the actuator lever in place as described above. The pump further comprises an inlet conduit 160 with an inlet valve 161 in fluid communication with the pump chamber, and an outlet conduit 170 with an outlet valve 171 in fluid communication with the pump chamber. The valves may be of any desirable configuration, but advantageously they are passive membrane valves.

FIG. 2A shows the pump and actuator assembly in an initial state with the actuator lever in an initial position in which the contact rod 137 is positioned against the first contact member 128 which thereby serves as a stop for the lever. As indicated above, the piston rod 151 has a length which ensures that it is forced by the pump membrane into contact with the lever in its initial position. The terms “initial” and “actuated” state refers to the shown embodiment in which the actuator is used to actuate the pump to produce a pump stroke, however, although the suction stroke of the pump may be passive (i.e. performed by the elastic energy stored in the pump membrane during the pump stroke) the actuator may also be actuated in the reverse direction (i.e. from the actuated to the initial position) to actively drive the pump during the suction stroke. Thus, in more general terms the actuator is moved between first and second positions in either direction.

FIG. 2B shows the pump and actuator assembly in an intermediate state in which the coil 136 has been energized (e.g. by a ramped PWM pulse) pivoting the lever relative to the first pivot joint 126, 133 thereby actuating the pump membrane via the piston 151, 155. As appears, the contact rod is now positioned between the two contact members 128, 129.

FIG. 2C shows the pump and actuator assembly in a fully activated state with the actuator lever in a fully actuated position in which the contact rod 137 is positioned against the second contact member 129 which thereby also serves as a stop for the lever. In this way the stroke distance and thus the stroke volume of the pump membrane is determined by the two contact (or stop) members 128, 129. In this position the coil is de-energized and the actuator lever is returned to its initial position by means of the biasing force of the pump membrane which during its travel to its initial position performs a suction stroke. If desirable, the actuator lever may also be returned to its initial position actively by reversing the current flow in the coil, however, in order to keep the actuator rod and the lever in contact with each other, this actuation should not be too swift.

FIG. 3A shows an alternative embodiment in which the actuator lever comprises two knife-edge members 233, 234 which cooperate with substantially planar surfaces on the housing support 226 and the free end 252 of the piston 251 to provide first and second pivoting joints. By this arrangement the distance between the two pivoting points, and thus the piston stroke length, is determined by properties of the lever which is allowed to “float” with respect to the two planar joint surfaces. Indeed, the housing should be provided with appropriate stops (not shown) preventing the lever from dislocating out of engagement. Further, two contact members 228, 229 are arranged on the lever cooperating with a contact rod 237 mounted on the housing, the opposed surfaces of the rod thereby serving as first and second stop means adapted to engage the actuator member in the initial respectively the actuated position. In this way the rotational freedom of the lever relative to the first pivoting joint, and thus the piston stroke length, is determined by the position of the contact members and the diameter of the contact rod. As appears, by this arrangement the structures most important for controlling the stroke length of the piston are all provided as parts of the lever. In an alternative embodiment (corresponding to FIG. 1) the housing support 226 comprises a groove in which the first knife-edge member 233 is located. In this way the lever is no longer allowed to “float”, however, due to the planer surface 252 on the piston, the stroke length is controlled by the position of the knife-edge members and not the precise position of the piston relative to the housing support groove. A non-floating joint between the housing and the lever is not limited to a knife-edge joint but may have any desirable configuration, e.g. a film hinge joint. Further, the line-contact joint provided by a knife-edge joint may be replaced by a punctual-contact joint provided by e.g. a spherical member resting on a planar surface. In the shown embodiment two pair of conductors 238, 239 are supplied to the coil respectively the contact members, however, alternatively the contact members may be connected to the coil conductors which then may serve to both energize the coil and conduct contact information to a processor or control system (not shown). For example, in case the contact rod is provided with a given resting voltage this voltage will change as the coil is energized with the contact rod in contact with the first contact member 229 and will change again as the second contact member 228 is moved into contact with the contact rod.

In the FIGS. 2 and 3 embodiments the piston-lever joint is provided between the housing-lever joint and the actuator coil, however, the positions may also be reversed such that the housing-lever joint is arranged between the piston-lever joint and the coil (not shown).

In FIGS. 2 and 3 the rotational (pivoting) freedom for the actuator lever has been provided by structures associated with the lever, however, in an alternative embodiment shown in FIG. 4 the structures controlling rotational lever movement and providing contact information are associated with the piston rod. More specifically, the piston rod 351 guided in a bore 356 comprises first and second collar members 358, 357 forming a gap in which a stop member 380 connected to the pump housing is arranged. In this way piston stroke length is determined by the thickness of the stop member and the distance between the two collar members. In the shown embodiment the two collar members are formed from metal and cooperate with a pair of conductors 381 arranged on the stop member.

With reference to FIG. 5 a further pump actuator will be described. Although the figure is oriented differently, the same terminology as for FIG. 1 will be used, the two pump actuators generally having the same configuration. The pump actuator 500 comprises an upper housing member 510 and a lower housing member 520, both comprising a distal main portion 511, 521 and a there from extending proximal arm portion 512, 522. Extending from the lower main portion a pair of opposed connection members 523, 524 are arranged, and at the proximal end of the lower arm a proximal connection member 525 is arranged perpendicularly to the general plane of the lower arm, the proximal connection member serving as a mount for a slotted joint mount 527. Further, a separate proximal connection member 526 is provided. In an assembled state the two main portions and the proximal connection member form a housing in which two pair of magnets 540, 541 are arranged on the opposed upper and lower inner surfaces of the main portions. The pump actuator further comprises a lever 530 having a proximal end 531 comprising first and second longitudinally offset and opposed joint structures in the form of an axle rod 533 respectively a joint rod 534 arranged perpendicular to a longitudinal axis of the lever, and a distal end 532 with a pair of gripping arms 535 for holding a coil member 536 wound from a conductor. A membrane pump (not shown) comprises an actuation/piston rod 551 is arranged, the piston rod serving to actuate the pump membrane of the membrane pump. The outer free end of the rod is configured as a substantially planar surface 552. The actuator further comprises a pair of rod-formed contact members 528, 529 mounted on the distal end of the lever and adapted to cooperate with a contact rod 537 mounted in the proximal connection member. Although the two joint rods 533, 534 and the contact members 528, 529 are shown as separate members, they are preferably all metallic members moulded into a lever formed from a polymeric material.

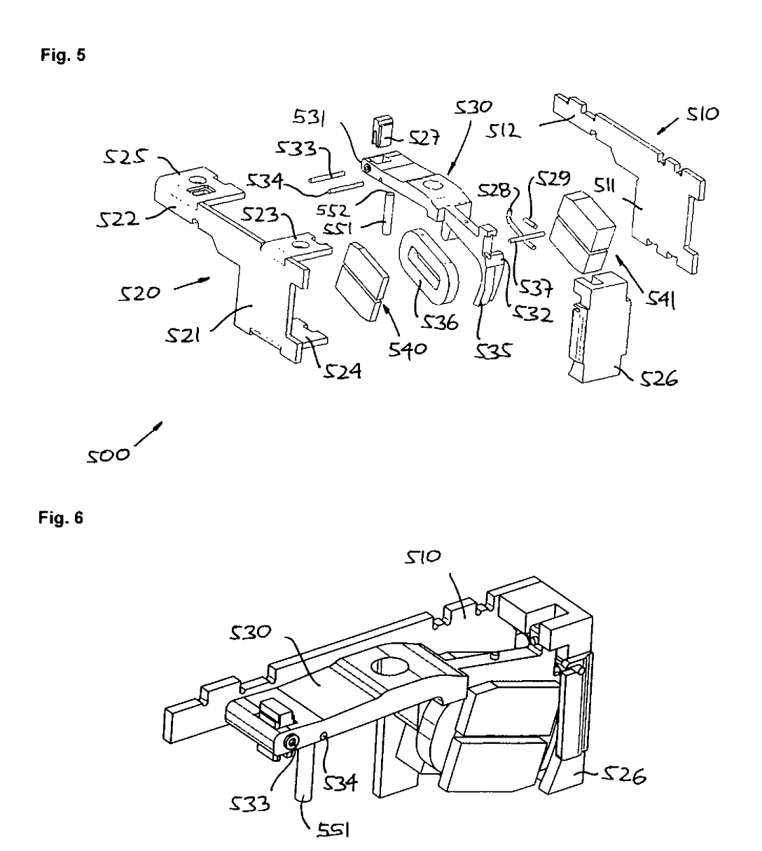

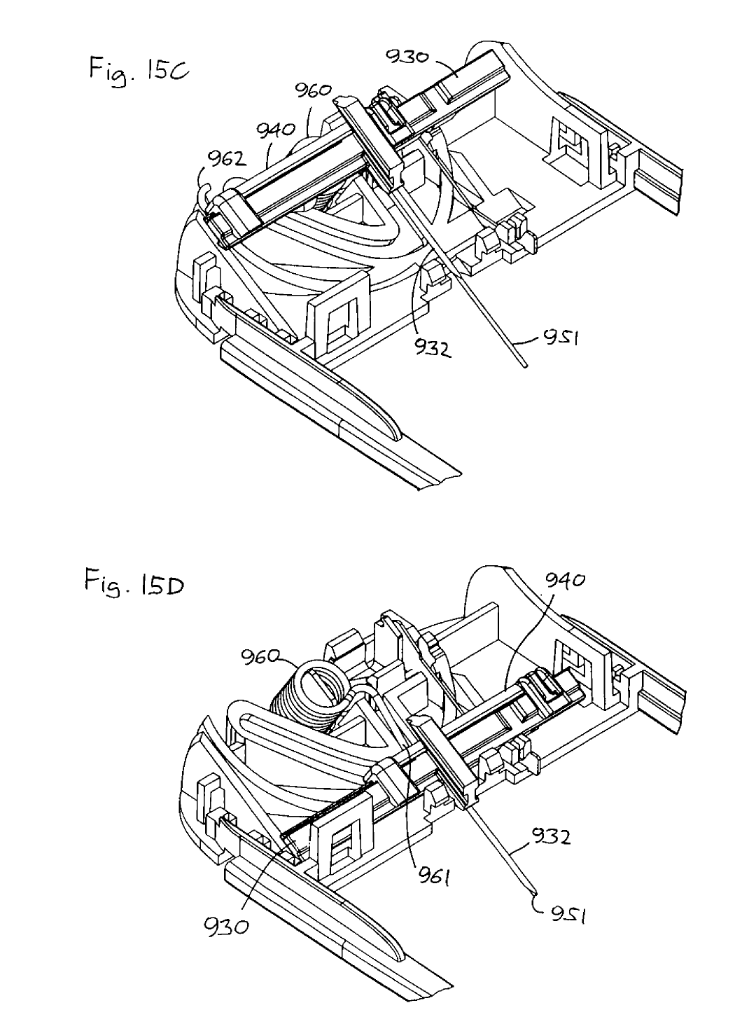

In an assembled state as shown in FIG. 6 (the lower housing member not being shown for clarity reasons) the lever is arranged inside a housing formed by the upper and lower housing members and the proximal connection member, with the coil positioned between the two pair of magnets. The axle rod 533 is arranged in the slotted joint mount thereby forming a proximal pivot joint. When the actuator is attached to a pump assembly (see e.g. FIG. 16) the joint rod 534 engages the substantially planar end surface 552 of the piston rod, thereby forming a distal floating knife-edge pivot joint. Although the joint rod is not a “knife”, the circular cross-sectional configuration of the rod provides a line of contact between the rod and the end surface, and thus a “knife-edge” joint. Using a more generic term, such a joint may also be termed a “line” joint. Due to this arrangement a gearing of the force provided from the coil-magnet actuator to the actuation rod is realized, the gearing being determined by the distance between the two pivot joints and the distance between the proximal pivot joint and the “effective” position of the coil on the lever. As the piston rod is biased outwardly by the elastic pump membrane the lever is held in place by the two joints and the housing in combination, the lever only being allowed to pivot relative to the first joint (see also below).

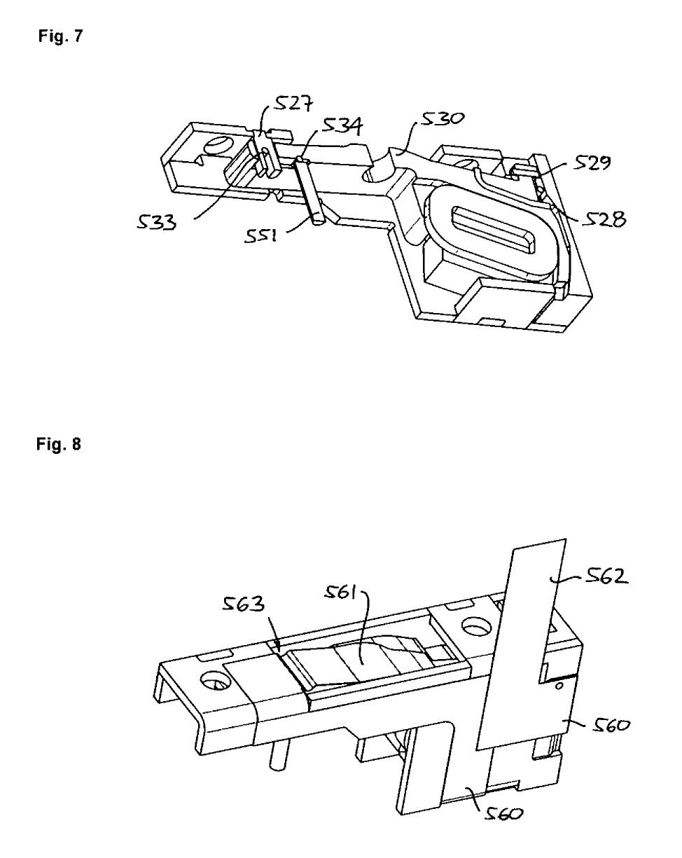

In the cross-sectional view of FIG. 7 it can be seen how the axle rod 533 is arranged in the slotted joint mount 527 (e.g. by snap-action) to form a pivot joint (which in the shown configuration may also be termed a bearing), and how the joint rod 534 engages the free end of the piston rod 551 to form a floating knife-edge pivot joint. Further, the contact members 528, 529 embedded in the lever 530 can be seen.

In order to provide electrical connections between the electrical components of the actuator, i.e. the contact members and the coil, and controller circuitry (see FIG. 16) the assembled actuator is provided with a flex print as seen in FIG. 8. The flex print comprises a main portion 560 mounted to the housing of the actuator, a lever portion 561 mounted to the lever, and a connecting portion 562 providing connection with the controller electronics. A film hinge 563 is provided between the main portion and the lever portion, this allowing the lever to pivot substantially freely. The flex print may be attached by any suitable means, e.g. adhesives or mechanical connectors.

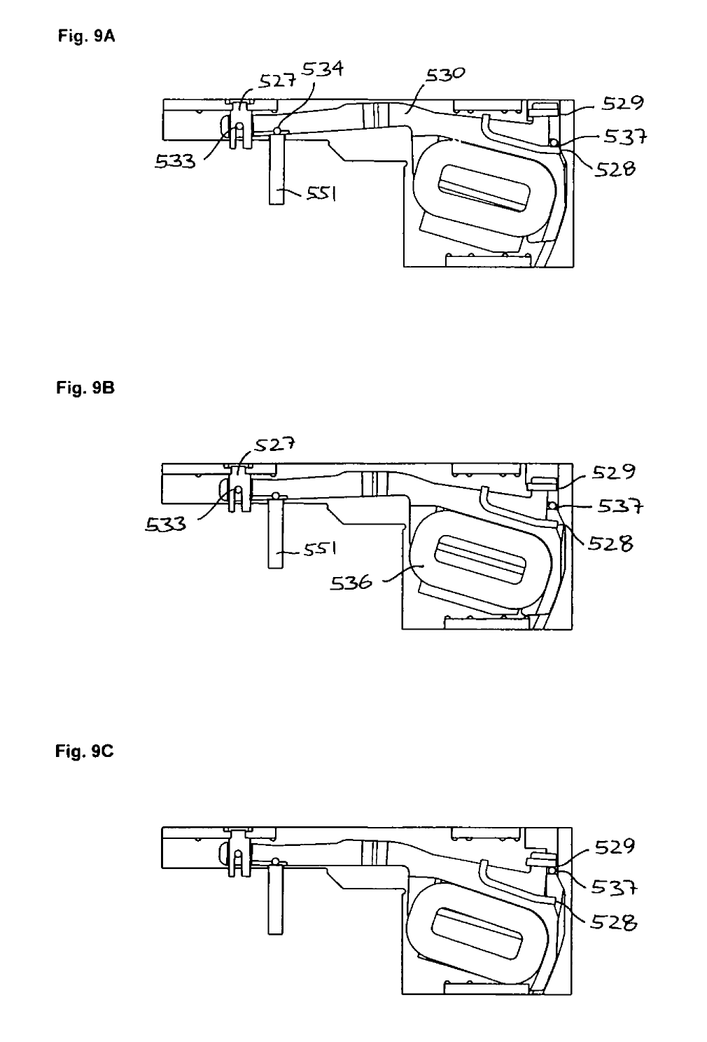

FIGS. 9A-9C show schematic cross-sectional views through an actuator assembly of the type shown in FIG. 5, the sections corresponding to a plane through the lever. The actuator is shown in an engagement with a piston rod 551 of a membrane pump (not shown) of the same principle configuration as shown in FIG. 2A. The pump membrane is in all positions in a stretched state, the membrane thereby exerting a biasing force on the piston rod which is used to hold the actuator lever in place as described above.

FIG. 9A shows the piston rod and actuator assembly in an initial state with the actuator lever in an initial position in which the contact rod 537 is positioned against the first contact member 528 which thereby serves as a stop for the lever. A proximal non-floating pivot joint is formed between the axle rod 533 and the slotted joint mount 527, and a distal floating pivot joint is formed between the joint rod 534 and the upper end of the piston rod 551. By this arrangement the distance between the two pivot points, and thus the piston stroke length, is determined by properties of the lever, whereas the lever and the piston rod is allowed to “float” with respect to each other. Further, the two contact members 528, 529 arranged on the lever cooperate with the contact rod 537 mounted on the housing, the opposed surfaces of the rod thereby serving as first and second stop means adapted to engage the actuator member (here: the lever) in the initial respectively the actuated position. In this way the rotational freedom of the lever relative to the first pivot joint, and thus the piston stroke length, is determined by the position of the contact members and the diameter of the contact rod. As appears, by this arrangement the structures most important for controlling the stroke length of the piston are all provided as parts of the lever. As indicated above, the piston rod 551 has a length which ensures that it is forced by the pump membrane into contact with the lever in its initial position. As for the embodiment of FIGS. 3A-3C the terms “initial” and “actuated” refers to the shown embodiment in which the actuator is used to actuate the pump to produce a pump stroke.

FIG. 9B shows the actuator assembly in an intermediate state in which the coil 536 has been energized pivoting the lever relative to the proximal pivot joint 533, 527 thereby actuating the pump membrane via the piston 551. As appears, the contact rod is now positioned between the two contact members 528, 529.

FIG. 9C shows the actuator assembly in a fully activated state with the actuator lever in a fully actuated position in which the contact rod 537 is positioned against the second contact member 529 which thereby also serves as a stop for the lever. In this way the stroke distance and thus the stroke volume of the pump membrane is determined by the two contact (or stop) members 528, 529. In this position the coil is de-energized and the actuator lever is returned to its initial position by means of the biasing force of the pump membrane which during its travel to its initial position performs a suction stroke. If desirable, the actuator lever may also be returned to its initial position actively by reversing the current flow in the coil.

As appears from the above, the two contact/stop members serve to control the stroke volume of the pump, however, they may also be used to control operation and performance of the actuated component (e.g. a pump) and the system/device in which it is embedded. More specifically, such information can be retrieved by detecting the time lapsed for moving the lever between its initial and actuated position. In the following this principle will be illustrated by means of a skin-mountable drug delivery device comprising a drug-filled reservoir, a pump and a transcutaneous access device. Before turning to the control system, an illustrative drug delivery device will be described in detail.

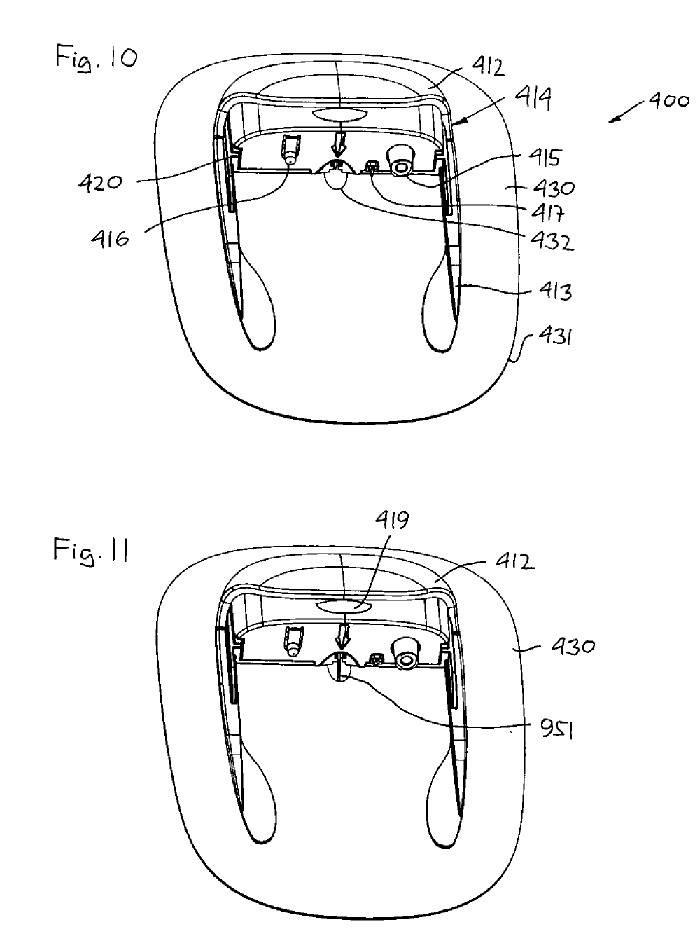

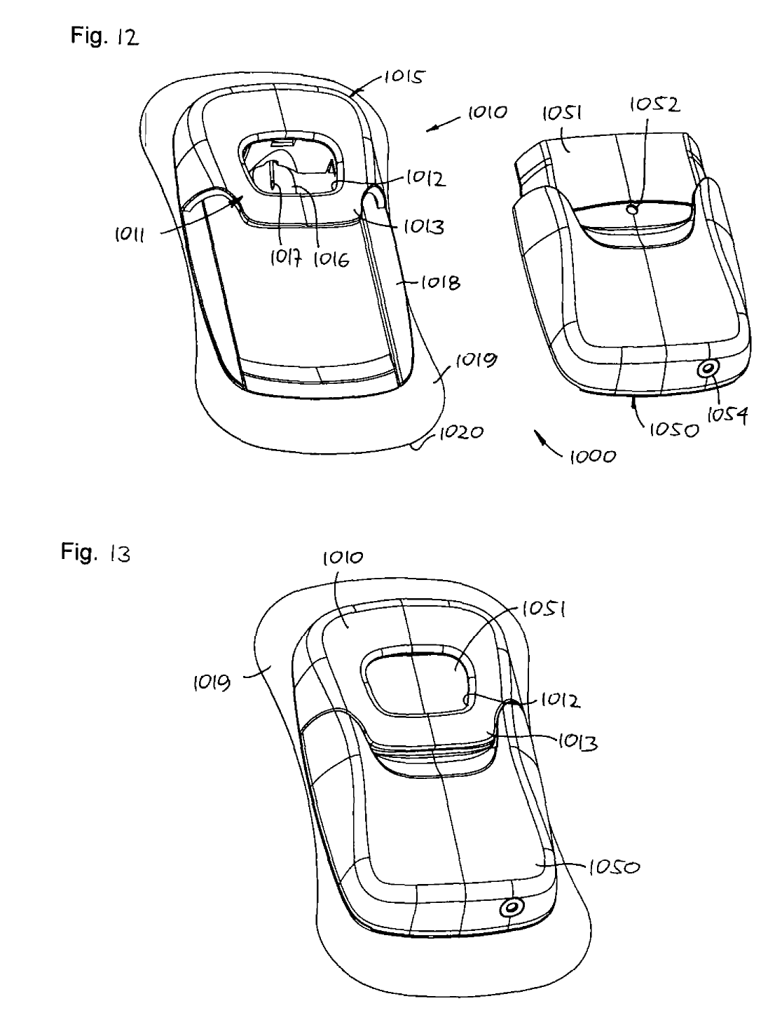

FIG. 10 shows a skin-mountable device in the form of a patch (or cannula) unit 400. The patch unit comprises a relatively rigid body portion 414 arranged on a flexible sheet member 430 with a lower mounting surface 431 provided with an adhesive allowing the sheet to be adhered to a skin surface of a subject. The sheet member comprises a central opening 432 through which a cannula can be inserted. The body portion comprises a housing portion 412 in which a cannula inserting mechanism is arranged, see below. The body portion further comprises two slider leg members 413 extending from the housing, the legs adding stiffness to the patch and further serves as guiding means when a pump/reservoir unit is attached the patch unit, see below. The housing is provided with a set of opposed grooves 420 serving as attachment means for a packaging and subsequently for a pump unit. The housing further comprises a fluid inlet 415 adapted to be mounted in fluid communication with a corresponding fluid outlet from an attached pump unit 450, an actuator 416 for actuating an electrical contact on the attached pump, and a release member 417 adapted to release a cannula inserting mechanism when the pump unit is attached for the first time, the cannula being inserted through the opening 432. The housing portion 412 also comprises a catch 419 adapted to engage a corresponding coupling structure on the pump unit. As appears, when the cannula 951 is inserted (see FIG. 11), it is protected by the pump unit, however, the pump unit can be removed for subsequent inspection of the insertion site as shown in FIG. 12.

FIG. 12 shows an alternative embodiment of a patch unit 1010 with a pump unit 1050 by its side, and FIG. 13 shows the pump unit fully but releasably attached. More specifically, FIG. 12 shows an embodiment of a medical device 1000, comprising a cannula unit 1010 of the type shown in FIG. 10 and a thereto mountable pump (or reservoir) unit 1050. In the shown embodiment the cannula unit comprises a housing 1015 with a shaft into which a portion 1051 of the pump unit is inserted. The shaft has a lid portion 1011 with an opening 1012, the free end of the lid forming a flexible latch member 1013 with a lower protrusion (not shown) adapted to engage a corresponding depression 1052 in the pump unit, whereby a snap-action coupling is provided when the pump unit is inserted into the shaft of the cannula unit. Also a vent opening 1054 can be seen. The housing 1015 is provided with a pair of opposed legs 1018 and is mounted on top of a flexible sheet member 1019 with a lower adhesive surface 1020 serving as a mounting surface, the sheet member comprising an opening 1016 for the cannula 1017.

As appears, from the housing of the cannula unit extends a cannula at an inclined angle, the cannula being arranged in such a way that its insertion site through a skin surface can be inspected (in the figure the full cannula can be seen), e.g. just after insertion. In the shown embodiment the opening in the lid provides improved inspectability of the insertion site. When the pump unit is connected to the cannula unit it fully covers and protects the cannula and the insertion site from influences from the outside, e.g. water, dirt and mechanical forces (see FIG. 13), however, as the pump unit is detachable connected to the cannula unit, it can be released (by lifting the latch member) and withdrawn fully or partly from the cannula unit, this allowing the insertion site to be inspected at any desired point of time. By this arrangement a drug delivery device is provided which has a transcutaneous device, e.g. a soft cannula as shown, which is very well protected during normal use, however, which by fully or partly detachment of the pump unit can be inspected as desired. Indeed, a given device may be formed in such a way that the insertion site can also be inspected, at least to a certain degree, during attachment of the pump, e.g. by corresponding openings or transparent areas, however, the attached pump provides a high degree of protection during use irrespective of the insertion site being fully or partly occluded for inspection during attachment of the pump. In the shown embodiment an inclined cannula is used, however, in alternative embodiments a needle or cannula may be inserted perpendicularly relative to the mounting surface.

FIG. 14 shows in an exploded view a drawing of a schematic representation of a patch unit (here a cannula unit) comprising a mechanism for inserting a soft cannula. More specifically, the unit comprises a bottom part 910 onto which is mounted a chassis part 920 thereby creating an interior in which the different parts of the mechanism are arranged. In addition to the functional portions of the bottom and chassis part the mechanism comprises a needle holder 930 with a needle mount 931 to which a needle 932 is mounted, a cannula holder 940 comprising first and second gripping portions 941, 942 adapted to engage the needle holder, and a hollow cannula assembly comprising a soft, flexible cannula with a distal portion 951, an intermediate portion 952, and a proximal portion 953, the cannula assembly further comprising a tubular housing member 955 adapted to engage an opening 922 in the chassis portion, an elastomeric tubular member 956 in which the proximal end of the cannula is mounted, and a needle pierceable elastomeric septum, the tubular member and the septum being arranged in the housing member thereby providing a fluid inlet port for the hollow cannula. The mechanism further comprises a coil-formed torsion spring 960 comprising an actuator arm 961 with a curved distal end 962, the spring being arranged in a spring holder 970 comprising a catch 971 allowing the spring to be mounted in a pre-tensioned state. A release member 975 is provided comprising an outer end portion 976 adapted to engage e.g. a pump unit when the latter is mounted, and an inner end portion 977 adapted to engage and release the actuator arm from the spring holder. The bottom part comprises an inclined surface 914. with a guide 912 comprising a first guide groove 913 arranged corresponding to a longitudinal axis of the unit, and a second guide groove 914 arranged at an angle of 45 degrees relative to the first guide groove.

In the assembled state the cannula holder is mounted on the needle holder with the gripping portions 941, 942 arranged on each side of the needle mount 931, this allowing the cannula holder to slide along the length of the needle holder, the two holders thereby forming an inserter. In an initial state the distal portion of the cannula is positioned in the needle and the intermediate portion is positioned in a channel formed between the needle holder and the cannula holder, the cannula being mounted to the cannula holder by means of a flexible member on the first gripping portion.

In the assembled state the needle holder with the cannula holder mounted is arranged on the inclined surface and is allowed to slide up and down, with the guide grooves adapted to engage a guide member arranged on the lower surface of the cannula holder (not shown). To control movement of the needle holder the needle mount comprises a guide portion 933 with two opposed grooves adapted to engage a corresponding guide member 921 arranged on an interior surface of the chassis part. As appears, in the shown schematic drawing the indined surface 914 is shown without cut-out portions allowing the release member 975 and the spring holder 970 to be mounted (see below).

The bottom part 910 further comprises two opposed leg portions 918 each with a lobe 919, the lobes providing attachment points when the bottom part is mounted to a flexible sheet or foil member 901 comprising an adhesive lower mounting surface 904 allowing the transcutaneous unit to be mounted on a skin surface of a subject. The sheet member comprises a central opening 903 through which the needle and cannula is introduced, as well as a release liner 902. A cover portion 905 serves to close the interior thereby forming a substantially closed housing.

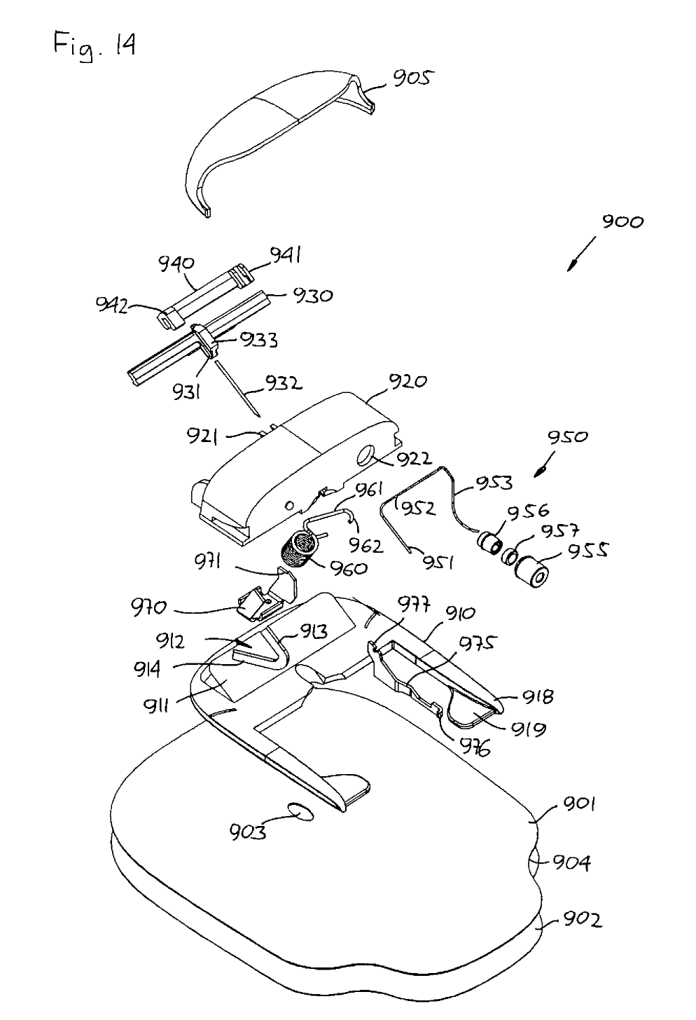

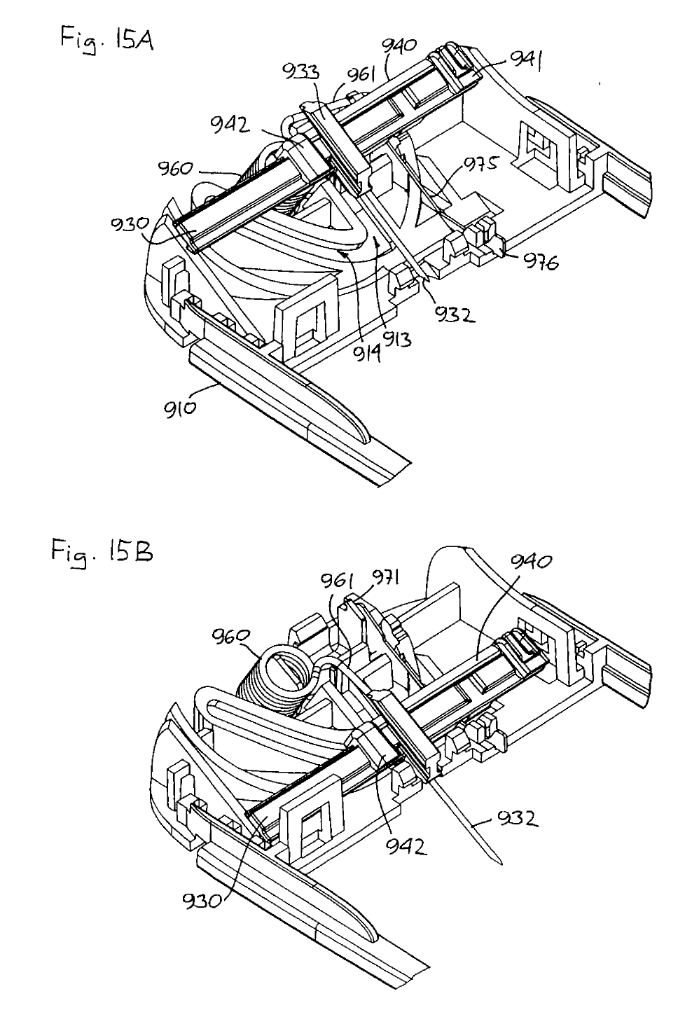

With reference to FIGS. 15A-15D the mechanism described with reference to FIG. 14 is shown in a partly assembled state, the chassis part and the proximal portion of the cannula not being shown. The assembled embodiment differs slightly from the above-described embodiment, however, as the differences are small the same reference numerals are used.

The assembled embodiment primarily differs from the FIG. 14 embodiment in that the inclined surface 914. has been replaced with a number of wall members, the upper surfaces of these wall members in combination providing an inclined “surface” on which the needle holder is arranged, this allowing the spring 960 and release member 975 to be shown functionally correctly arranged.

FIG. 15A shows the assembly in an initial state with the needle holder 930 in a first (or initial) retracted position with the needle correspondingly in its retracted position with the distal pointed end arranged within the housing. The cannula holder is positioned in a right-most position on the needle holder corresponding to its retracted position. The distal portion of the cannula is positioned in the needle with the distal end just within the distal end of the needle, and the intermediate portion is positioned in the channel formed between the needle holder and the cannula holder, the cannula being gripped by a flexible arm formed as part of the first gripping member 941.

When a pump unit (not shown) is attached to the cannula unit the pump unit engages and pushes the outer end portion 976 of the release member 975, thereby releasing the spring actuator arm 961. The actuator then starts to turn clockwise (as seen in the figure) and engages a rear surface of the needle member pushing it forward to its extended position as seen in FIG. 15B. During this movement the needle holder is guided linearly by engagement with the guide member 921 arranged on an interior surface of the chassis part, whereas the cannula correspondingly is guided linearly to its first extended position by engagement with the first guide groove 913. Thus, during this forward movement, the cannula holder does not move relative to the needle holder.

In this position the needle holder cannot be moved further forward, and as the spring actuator arm continues to turn clockwise it engages the guide member arranged on the lower surface of the cannula holder (not shown) thereby starting to move the cannula holder to the left, sliding on the needle holder. At this position the guide member has reached the lower end of the first guide groove (see FIG. 14) and is now moved into the second inclined guide groove where it is moved upwards along the guide groove, thereby being moved further to the left. As the cannula holder is attached to the needle holder, the needle holder is also moved upwards, however, it is guided linearly backwards due to the engagement with the guide member 921. When the cannula holder has reached the upper end of the second guide groove, it has reached its second extended position just as the needle holder has reached its second retracted position (the first and second retracted positions may be the same), just as the cannula holder has reached its second extended position.

As described above, the cannula has a distal portion initially arranged within the needle, an intermediate portion arranged in the channel formed between the cannula and needle holder, and a proximal portion serving as a flexible connection between the moving inserter and the fluid inlet port. As the cannula is attached to the cannula holder corresponding to the proximal end of the intermediate portion, movement to the left of the cannula holder will push the cannula through the channel, around the bend connecting the channel and the needle, and down into the needle. Thus as the cannula holder is moved from its first to its second extended position, the cannula is pushed out through the needle, whereas in the meantime the needle holder with the needle is retracted (see FIG. 15C). In case the cannula and needle are extended respectively retracted at the same speed (this corresponding to the second guide groove being straight and arranged at an angle of 45 degrees relative to the first guide groove) then the distal portion of the extended cannula will not move relative to the housing, whereas the needle will be retracted.

In order to allow the guide member of the cannula holder to properly enter the second guide groove, it may be desirable to connect the two guide grooves with a short groove portion, this providing that the cannula will be extended a little before the needle starts to retract, this as shown in FIG. 15D. Correspondingly, by modifying the configuration of the second guide groove it is possible to retract the cannula a little from its most extended position. The latter may be desirable in order to free a distal cannula opening from any tissue plug formed during insertion.

FIG. 16 shows in an exploded view a pump unit 300 of the same type as in FIG. 12. The pump unit comprises an upper housing portion 310 and a lower housing portion 320 which in an assembled state provides a water-protected enclosure for the additional components of the reservoir unit: A pump assembly 330, an actuator 340, a reservoir 350, and electronic control means 360. In an initial state as supplied to the user, a protective cap assembly 370 is attached to the unit.

The lower housing portion is made from a transparent material allowing a reservoir (see below) to be inspected by a user from the outside, and comprises an opening 321 in which a water repelling vent 322 is arranged. A sheet member 325 with a window opening 326 is attached to the lower surface of the lower housing portion, this masking the transparent portion except for a window over the reservoir. The sheet member may be used to display user information, e.g. type and amount of drug.

The pump assembly 330 is in the form of a membrane pump comprising a piston-actuated pump membrane with flow-controlled inlet- and outlet-valves. The pump has a general layered construction comprising a number of body members between which are interposed flexible membrane layers, whereby a pump chamber, inlet and outlet valves, and one or more safety valves can be formed, the layers being hold together with clamps 338. The pump further comprises a fluid connector 335 in the form of hollow connection needle slidably positioned within the pump (for illustrative purposes shown outside of the pump), this allowing the pump to be connected with reservoir when the protective cap assembly 370 is activated. For a more detailed description of such a membrane pump reference is made to applicants co-pending application PCT/EP2006/060277, which is hereby incorporated by reference.

The pump actuator is in the form of a coil actuator to which the pump assembly is attached by a clamp. For a more detailed description of such a coil actuator reference is made to the description of FIGS. 1-9 above and applicants co-pending application WO 2005/094919, which is hereby incorporated by reference.

The drug reservoir is in the form of a flexible, pre-filled collapsible pouch 350 comprising a needle-penetratable septum 354 allowing the fluid connector to be pushed into the reservoir without leakage, thereby providing a fluid communication with the pump. A clip holder 352 is attached to the reservoir, this allowing the reservoir to be attached to the housing without influencing the reservoir per se. Under the reservoir (as seen from the lower surface of the unit) is arranged a sheet (not shown) comprising a contrast-enhancing pattern, e.g. a black line on a white background, allowing for easier visual identification of impurities in the drug, e.g. fibrillation in insulin.

The electronic control means 360 comprises a PCB or flex-print 362 with a processor 361 for controlling the pump assembly, a battery 366, an acoustic transducer 365 providing an alarm and communication interface with the user, as well as a contact mounted on the actuator allowing the control means to be activated by the user when taken into use for the first time (via the actuator 216). The control means may comprise a receiver and/or a transmitter allowing the reservoir to communicate wirelessly with a remote controller.

The protective cap assembly 370 comprises an attachment member 371 initially locked to the reservoir unit and an activation “push bufton” member 372 slidingly attached to the attachment member. When the reservoir unit is removed from its primary packaging (not shown) the user depresses the activation member towards the reservoir unit. This actuation results in three actions taking place: A first protrusion on the activation member will actuate a contact on the reservoir unit, this activating the electronics, and a second protrusion will engage the pump assembly and push the fluid connector 335 out from the pump assembly and into the reservoir, thereby establishing a fluid communication between the reservoir and the pump. Thirdly, depression of the activation member will “unlock” the attachment member and allow it, and thereby the activation member, to be removed from the reservoir unit. Thereafter the reservoir unit can be connected to the patch unit.

Turning to the above-mentioned operation and performance control by means of elapsed time detection for actuator lever movement between an initial and an actuated position or vice versa, FIG. 17 shows a flow chart illustrating the sequence of operations carried out for an implementation of this principle. More specifically, signals provided from sensors or switches adapted to detect that an actuator member (e.g. a lever as in FIGS. 1-9) or a component functionally coupled to the actuator such as the above-described piston which is considered a part of the actuator although it may be integrally formed with the pump) has reached its initial respectively actuated position during an actuation cycle is fed to a processor (e.g. microprocessor). The sensors/switches may be of any suitable type, e.g. electrical, optical or magnetic. If the initial and/or the actuated position cannot be detected, the processor detects an error condition which may be related to the type of non-detection. For example, when the actuator is used for the first time, non-detection of one or both signals may be indicative of an inherent fault in the actuator/pump/device and a corresponding alarm condition may be initiated. In most cases it will be relevant to define a time window within which the two positions have to be detected during an actuation cycle, this in respect of both the actuation movement between the initial and actuated position and the return movement between the actuated and initial position. Correspondingly, if the time lapsed between the detection of an initial-to-actuated or actuated-to-initial movement falls outside the time window an alarm condition indicating a malfunctioning may be initiated as will be described in the following with reference to a number of examples. When calculating the time lapsed this may be based on two “real time” time stamps or a timer may be used when movement between the two positions is initiated.

Turning to “normal” operation conditions, the lapsed time for movement between the initial and the actuated position (or between the actuated and the initial position) is calculated and compared with set time value ranges (e.g. pre-set or calculated ranges). Depending on the relation between the time lapsed and the set time value ranges a given pre-defined signal (or non-signal) is output from the processor which may then be utilized to perform a given action relevant for the device or system in which the actuator and control system is implemented.

Whereas a general example of an actuator operation and performance control principle has been described above, a more specific implementation of the principle will be described with reference to a drug delivery device of the type described above.

During operation of the pump after priming of an initially empty pump, liquid drug is sucked from the flexible reservoir into the pump chamber as the piston/actuator returns from an actuated to an initial position, whereas liquid drug is pumped from the pump chamber out through the transcutaneous access device as the piston/actuator is moved from the initial to the actuated position. During normal operation of the pump the time used for both of these pump strokes can be assumed to be near-constant as the conditions remain substantially unchanged. However, during operation of the pump certain conditions may arise which will influence operation of the pump and thereby potentially also of the amount of drug delivered. A major concern associated with infusion of drugs is occlusion of the access device.

A problem with existing drug delivery pumps is their ability to detect occlusions, especially when the pump is used for low flow applications. The problem is caused by the combination of low flow and compliance of the pump as it can take several hours for a blocked pump to build up enough pressure before the occlusion detector gives an alarm. Many traditional delivery pumps are compliant because the reservoir is part of the pump mechanism and/or because the fluid passage from the pump to the point of delivery (e.g. the distal end of an infusion needle) is compliant.

Using a membrane pump as a suction pump in a drug delivery device, a hydraulically much stiffer system can be achieved as the reservoir is “behind” the pump. Correspondingly, by also paying attention to the compliance of the outlet portion of the system a very stiff system may be provided such that an eventual occlusion will give an instant pressure increase, making it possible to alarm the user of an occlusion significantly faster than with traditional pumps. However, instead of providing an additional pressure sensor, the present invention can utilize that occlusion downstream of the pump will result in longer pump cycles for the outlet stroke given the same force is applied from the pump membrane actuator.

A further condition that would be desirable to detect would be under-dosing due to backflow of drug to the reservoir during the expelling stroke in case of malfunctioning of the inlet valve, e.g. when drug particles are captured in the valve. For such a condition it can be expected that the outlet stroke cycle will be shorter as a portion of the drug in the pump chamber is pumped backwards through the open inlet valve. In addition, this situation may also result in a shortened suction stroke as flow resistance through the open inlet valve may be reduced. On the other hand, in case of (partial) inlet valve occlusion, the suction stroke will result in longer cycle times. A longer suction stroke time may also be indicative of the reservoir being (close to) empty.

When a pump unit, e.g. as shown in FIG. 16, is supplied with both a sealed reservoir and a sealed pump, it is necessary to prime the pump with liquid drug when a new pump unit is connected to a patch unit for the first time. Correspondingly, when the pump controller detects this condition, a priming cycle is initiated. For example, the pump may be operated for a given number of cycles corresponding to the volume of the pump where after it is assumed that no gas remains in the pump. As gas has a much lower viscosity than a liquid drug, it can be assumed that a pump partially filled with air will have shortened cycle times for inlet and/or the outlet strokes. Correspondingly, by monitoring the cycle times during priming it can be controlled that the pump has been properly primed. For example, a priming cycle is started whereby the pump is actuated in accordance with a predetermined priming cycle frequency, and a first series of time lapsed values (in the following also time value or T) for movement of the pump membrane actuator associated with the pumping of a gas or a mixture of gas and liquid is detected. The detected time values are compared with a value associated with the pumping of a liquid. The latter may either be pre-defined or be calculated dynamically on the basis of the values detected by a series of pump strokes known to represent the pumping of air. In case the time values for a dry and a wet pump are similar, the controller may use another condition to determine that the pump has been properly primed, e.g. a rise in time values due to pumping of liquid though a restriction in the flow conduit down-stream of the pump, or due to the liquid entering the subcutaneous tissue of the user. In case the detected values (i.e. one or more) are within the pre-specified or calculated range, the priming cycle is ended. In case the detected values are not within the range, the priming cycle continues. In case the primed condition is not identified within a given pre-defined period, a malfunction condition can be identified. For the time values the suction stroke, the expelling stroke or both may be used as a basis for determining whether priming has taken place successfully. Alternatively, instead of comparing the detected time values with a preset or calculated specific value, it would also be possible to operate the pump until a steady state was achieved, i.e. the time pattern for a pre-defined number of operations vary within only a pre-defined range.

The processor should be adapted for compensating for “normal” bounce of the sensors/switches, however, excessive bouncing may be registered as a malfunctioning condition. Further, registering passive movement of the actuator during non-actuated periods may also be utilized to register a malfunctioning condition.

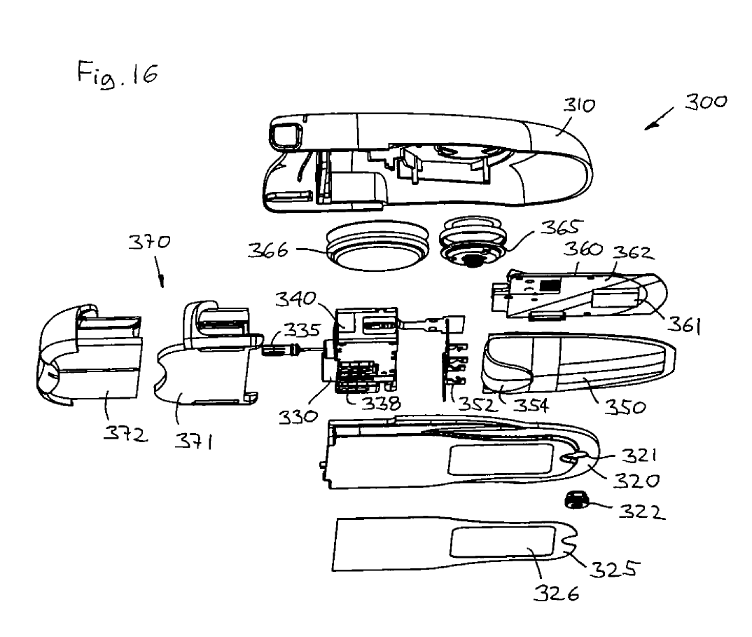

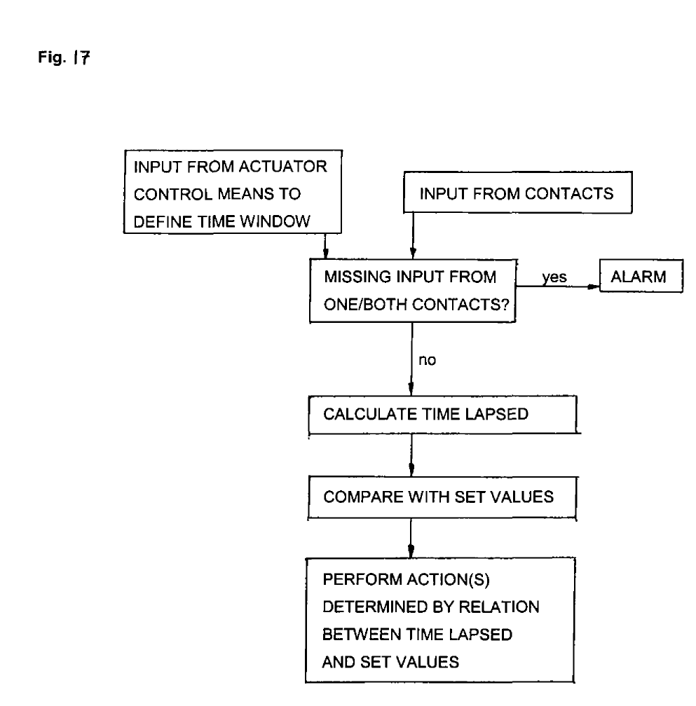

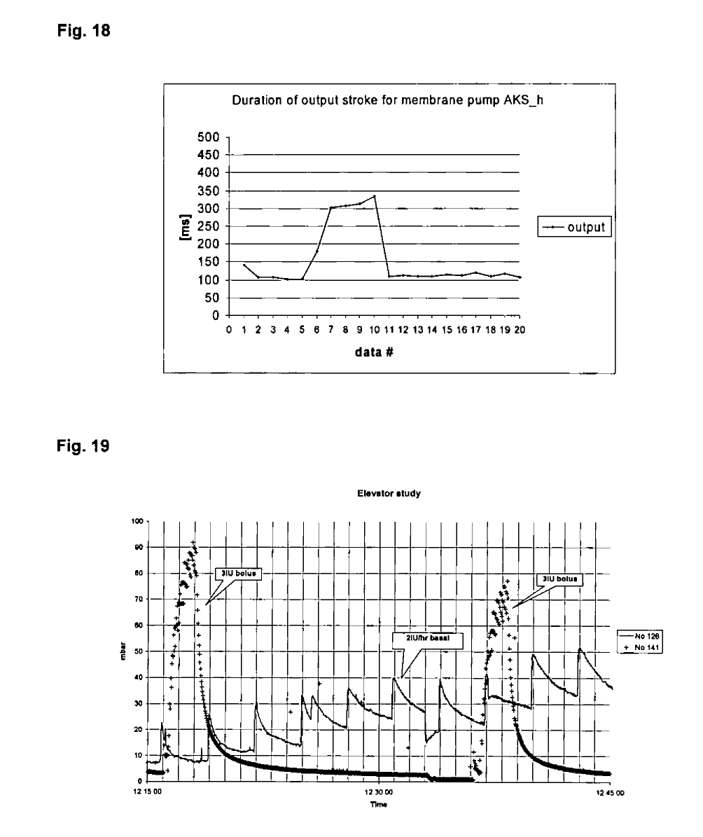

With reference to FIG. 18 an example based on an experiment conducted with a prototype version of the pump assembly shown in FIG. 16 will be described. Each data point represents an actuation of the coil actuator.

### Example 1

FIG. 18 shows the duration of an output stroke for an air filled membrane pump. At data # 5 the outlet conduit is occluded resulting in a higher counter pressure at the pump outlet. This pressure elevation results in a prolonged duration of the output stroke followed by a return to the previous duration when the occlusion was removed at data # 10. The experiment shows that the output stroke duration can be used as a measure of counter pressure during pump actuation. Correspondingly, it can be assumed that a higher flow resistance during subcutaneous infusion (see example 2 below) will result in prolonged duration of the output stroke as compared to a shorter duration during non-subcutaneous infusion, e.g. when a previously subcutaneously arranged transcutaneous access device is pulled out of the skin or otherwise displaced.

### Example 2

FIG. 19 shows data recorded in a pig subcutaneous infusion study with a MiniMed Pump and a pressure sensor in the catheter tube. The basal curve shows the pressure response of every 1 μl basal rate infusion and the bolus curve shows the pressure response of 30 μl bolus infusions. As appears, a considerable pressure is built up as fluid is infused subcutaneously during a bolus and, to a minor degree, at each pump actuation during basal rate infusion. The figure does not show the pressure in the catheter tube when the infusion catheter was removed from the pig, however, it can be assumed that the pressure rise will be significantly less and thus be indicative of non-subcutaneous delivery of liquid. Indeed, to discriminate between the above two situations, the pressure resistance in the conduit between the pump and the outlet of the transcutaneous access device should be relatively low as compared to the flow resistance in the subcutaneous tissue. As the pressure rise during bolus infusion is high, a single finding of a low flow resistance during bolus infusion may be indicative of non-subcutaneous infusion and thus trigger an alarm. However, the pressure rise during basal rate infusion is much lower, this making detection of the second condition during such infusions more difficult. Thus, if detection of the second condition is to be based upon basal rate infusions, the controller may be adapted to evaluate detected values and implement a “strategy” to avoid false positive determinations of the second condition, e.g. due to small variations in the flow resistance in the subcutaneous tissue as the user moves.

### Example 3

### Dynamic Range Calculation

Dependent upon the actual design of a given pump, it may be found that there is only minimal variation between the pumps and that substantially the same time values are detected when pumping. For such a pump design it may be desirable to use pre-set values, e.g. time ranges. However, for a different pump design there may be some variation between the individual pumps for which reason it may be desirable to calculate a set of ranges for the individual pump based on well-defined pump conditions. For example, when a new transcutaneous access device has been inserted (e.g. using a disposable drug delivery device with a build-in cannula, a disposable patch unit or a traditional infusion set) the pump is operated to properly prime the transcutaneous access device. As it can be assumed that the transcutaneous access device is properly in place in this situation, the values associated with pump actuation and detected during such priming operation can be used to determine a “subcutaneous state”. For example, the last 5 or 10 strokes during priming may be used to calculate an average “subcutaneous value” which then forms a basis for calculating an open range for defining a “non-subcutaneous state”. The “non-subcutaneous state” range may be defined by a factor, e.g. a T-out drop of 50% or more, or a numeric value, e.g. a T-out drop of 100 milliseconds (ms) or more. The “subcutaneous value” used for comparison may be calculated as an average of a number of individual values.

With reference to FIGS. 1-9 and 16 a reciprocating coil actuator adapted to be used in combination with a reciprocating membrane pump has been described, however, the present invention is also applicable in combination with other types of expelling assemblies and actuators.

With reference to FIG. 20 a further drug delivery device 600 will be described. The device comprises a housing 610, a cylindrical reservoir 620 with a piston 621 and a thereto attached plunger 622, an fluid outlet 630, a reciprocating actuator 640 (e.g. a coil actuator or a SMA actuator), and a drive mechanism 641 arranged between the actuator and the plunger for transforming the actuator input to a forwards movement of the plunger and thus the piston. The delivery device further comprises an energy source 661, an electronic controller 650 for controlling the actuator, the controller having a first sensor 651 for detecting movement of the actuator or mechanism, and a second pressure sensor 652 for detecting a pressure in the fluid outlet. The fluid outlet may be connected to an infusion set via a flexible tube or the device may be provided with a transcutaneous access device, either insertable or permanently protruding from the lower surface of the device. The shown device comprises two sensors adapted to detect different properties and which may both be used to perform aspects of the present invention, however, alternatively only one type of sensor may be used in a given drug delivery device. Incorporated US 2004/0127844 discloses a fluid delivery device comprising a pressure sensor.

In accordance with aspects of the present invention, one or both of the sensors 651, 652 can be used to detect a property (e.g. pressure in the fluid outlet or time lapsed for actuator movement) associated with operation of the actuator and delivery of fluid either subcutaneously or non-subcutaneously.

With reference to FIG. 21 a further drug delivery device 700 will be described. The device comprises a housing 710, a cylindrical reservoir 720 with a piston 721 and a thereto attached plunger 722, a fluid outlet 730 connected to an infusion set 731 via a flexible tube 732, a revolving motor 740, and a drive mechanism 741 arranged between the motor and the plunger for transforming the motor input to a forwards movement of the plunger and thus the piston. The delivery device further comprises an energy source 761, an electronic controller 750 for controlling the actuator, the controller comprising circuitry for detecting current supplied to the motor. An additional pressure sensor for sensing either the pressure in the fluid outlet or the pressure provided on the reservoir may be provided (not shown). Such a pressure sensor may be used in combination with the current sensor or as an alternative thereto. The fluid outlet may be connected to an infusion set via a flexible tube as shown or the device may be provided with a transcutaneous access device, either insertable or permanently protruding from the lower surface of the device. Incorporated U.S. Pat. No. 6,555,986 discloses a fluid delivery device comprising a current sensor.

The drug delivery device 700 further comprises a remote control unit 770 adapted to wirelessly communicate with the processor of the pump unit. The remote unit comprises a display 771 and user input keys 772, this allowing the remote unit to display information received from the pump unit (e.g. a visual and/or audible or tactile alarm indicating that the transcutaneous access device has disengaged from its subcutaneous position), and the user to enter flow control commands and instructions on the remote unit which is then transmitted to the pump unit. Indeed, such a remote control unit may also be used in combination with the above-disclosed pump units.

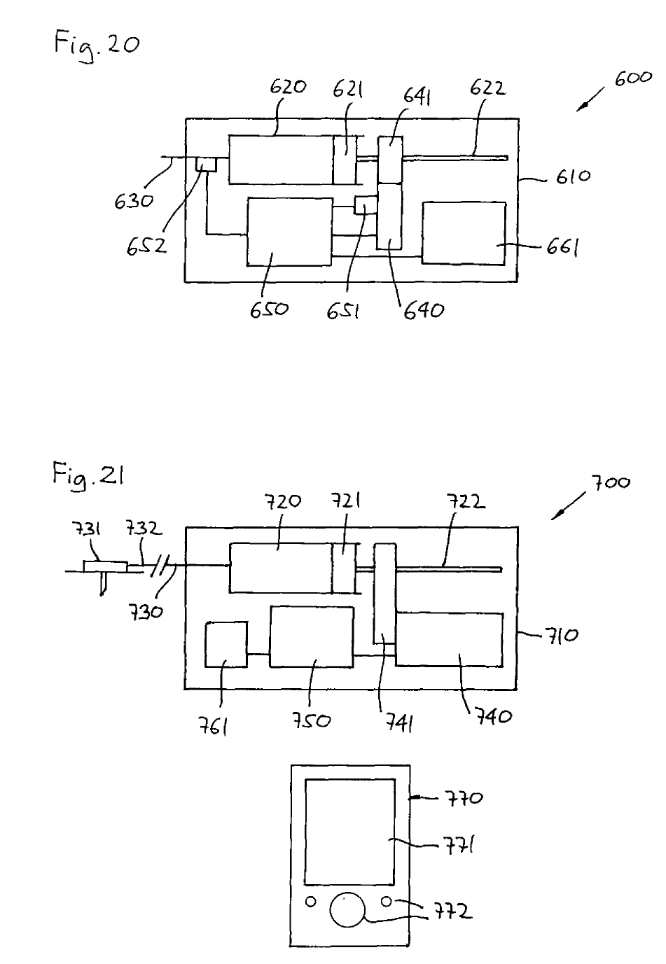

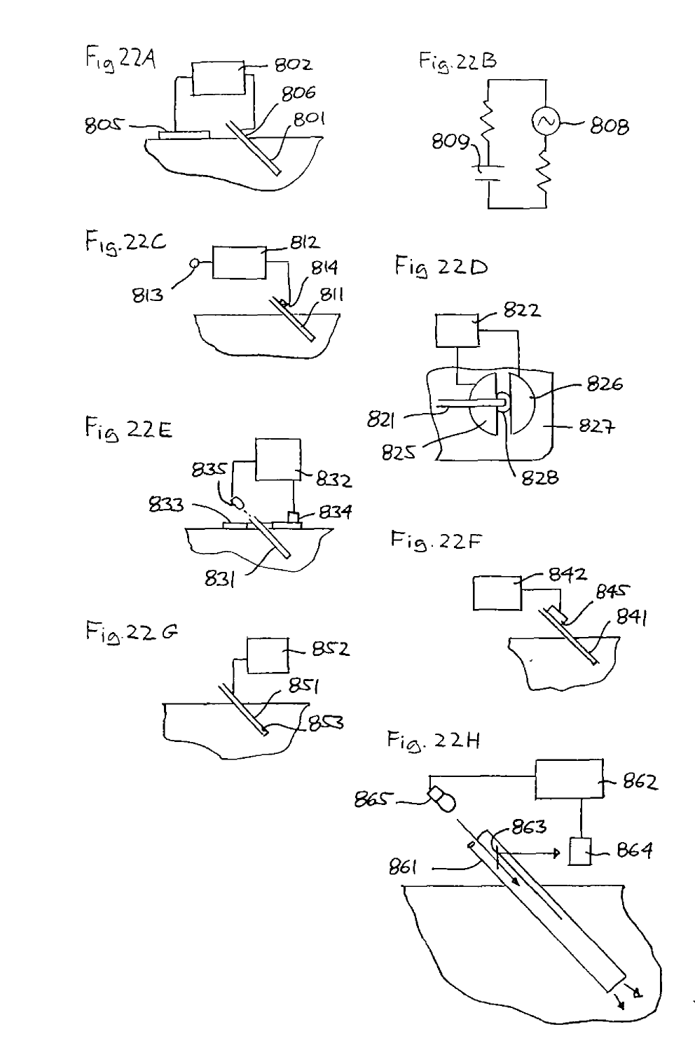

In the above embodiments detection of the position of a transcutaneous access device has been based on properties associated with the subcutaneous or non-subcutaneous delivery of drug, however, in accordance with the present invention the controller may be adapted to detect a first condition associated with actual subcutaneous placement of a transcutaneous device (e.g. a cannula or a sensor), respectively a second condition associated with non-subcutaneous placement of the transcutaneous device. With reference to FIGS. 22A-22H different embodiments of a medical device comprising a transcutaneous device and a processor as well as detection means for detecting placement of the transcutaneous device are shown. The figures are schematic and the body of the device as well as the additional structures related to infusion, e.g. reservoir and expelling assembly, are not shown, however, these may take the form of any of the above described embodiments.

FIG. 22A shows a subcutaneously arranged cannula 801 and a processor 802 connected to two electrodes 805, 806, the latter being formed by a conducting cannula or a fluid filled cannula wherein the drug serves as a conductor. The cannula may be made conductive by using a conductive polymer, e.g. a doped polymer, by a conductive inner or outer coating, or by providing a conductive trace. Correspondingly, the fluid drug should be properly conductive, e.g. by having a sufficient large number of free electrons. When an AC voltage is applied between the two electrodes the matter arranged between the electrodes will serve as a capacitance 809 as illustrated in the diagram shown in FIG. 22B. As the capacitance for subcutaneous tissue is different from air, it will be possible to detect whether the cannula is arranged subcutaneously or non-subcutaneously. This effect is substantial because the subcutaneous tissue operates as a low impedance plan (807) extending the one plate of the virtual plate capacitor to be very big. The other plate of the virtual plate capacitor is electrode 805. The capacity conducting the AC current is 808. When the cannula is out of the subcutaneous tissue the resulting capacity between the cannula electrode 806 and the electrode 805 is much smaller. The current could e.g. be measured over a resistance in the device (in processor 802).

FIG. 22C shows a subcutaneously arranged cannula 811 and a processor 812 connected to a first temperature sensor 813 arranged within the device and a second temperature sensor 814 arranged on the cannula which will serve as a conductor for heat from the subcutaneously tissue. As it can be assumed that the temperature of the cannula will decrease in case it disengages from its subcutaneous position, this can be used to detect whether the cannula is arranged subcutaneously or non-subcutaneously. Indeed, the temperature will be highest in the cannula just prior to a pump stroke in which the cannula is flushed and thus cooled with drug having the ambient temperature within the device.

FIG. 22D shows seen from above the skin a subcutaneously arranged cannula 821 and a processor 822 connected to two electrodes 825, 826 arranged on a flexible patch member 827 and on each side of an opening 828 through which the cannula is introduced through the skin, e.g. corresponding to the flexible patch member 432 of FIG. 10. In case the cannula disengages from a subcutaneous position, fluid drug will be pumped out on the surroundings and thus shorten the two electrodes, an event that will be detectable by the processor. The flexible patch member may also be provided with a marker substance that will change colour when subjected to contact with a given drug substance, e.g. insulin. Such a colour change may be detectable by a sensor connected to the processor 822 or by the naked eye of a user.

FIG. 22E shows a subcutaneously arranged light-conducting cannula 831, a processor 832, a ring-formed light conductor 833 comprising a light sensor 834 connected to the processor, and a light source 835 controlled by the processor. When light is directed through the cannula it will primarily exit at the distal end of the cannula and into the subcutaneous tissue, however, in case the cannula becomes arranged on the skin surface light from the distal end will be detectable by the sensor 834. Alternatively a number of light sensors may be used instead of the light conductor 833.

FIG. 22F shows a subcutaneously arranged cannula 841, a sound transducer 845 connected to the cannula, and a processor 842 controlling the transducer. The transducer is configured to both create and detect sound pulses (or vibration impulses). When the cannula is exited by application of an impulse, the impulse will travel along the length of the cannula, however, it will also travel back, the return signal then being detectable by the transducer. As it can be assumed that the sound-transmitting properties of the cannula will be influenced by the actual position of the cannula, this can be used to detect whether the cannula is arranged subcutaneously or non-subcutaneously.

FIG. 22G shows a subcutaneously arranged cannula 851 and a processor 852 connected to a temperature sensor 853 arranged at the distal end of the cannula. As it can be assumed that the temperature of the cannula will decrease in case it disengages from its subcutaneous position, this can be used to detect whether the cannula is arranged subcutaneously or non-subcutaneously.

FIG. 22H shows a subcutaneously arranged light-conducting cannula 861 within which is arranged a light splitter 863, a light sensor 864 connected to a processor 862, and a light source 865 controlled by the processor. When light is directed through the cannula and the therein contained fluid drug, it will primarily exit at the distal end of the cannula and into the subcutaneous tissue, however, some of the light will be reflected by the subcutaneous tissue and travel back through the cannula and fluid drug. When the light hits the light splitter it will be directed towards the light detector and thus measured. As it can be assumed that the amount of returned light will be influenced by the actual position of the cannula, this can be used to detect whether the cannula is arranged subcutaneously or non-subcutaneously.

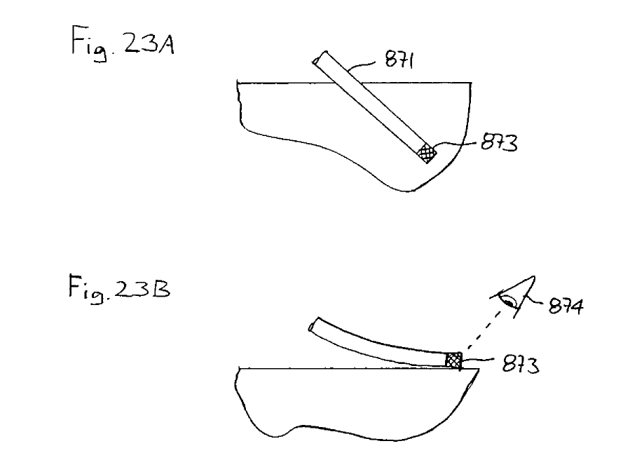

FIG. 23A shows a subcutaneously arranged flexible transcutaneous device 871, e.g. a cannula or sensor device, comprising a distal end with a visual indication 873, e.g. in the form of a ring marker having a colour such as red, black or blue which will be readily identifiable by the naked eye 874 of a user should the transcutaneous device disengage from its intended subcutaneous position. Indeed, such a marked transcutaneous device may be utilized in combination with any device carrying a transcutaneous device and where it is desirable to be able to check the position of the distal end of the transcutaneous device, e.g. for a medical device as described above or for a conventional infusion set. The visual marking may also be arranged along the length of the distal portion of the transcutaneous device. In a first alternative, the transcutaneous device comprises a colour marking arranged along the length of the cannula, whereby such a marking could be provided during manufacture of the transcutaneous device in running length, e.g. by co-extrusion or laser engraving for a cannula. In a second alternative, the transcutaneous device generally has a “signal” colour such as red, black or blue instead of the transparent, white or whitish colour inherent to PCTFE or other medical grade materials suitable for a soft cannula. All of these markings would make it easier for the user to detect whether the transcutaneous device is arranged on or below the skin surface.

In the above description of the exemplary embodiments, the different structures providing the described functionality for the different components have been described to a degree to which the concepts of the present invention will be apparent to the skilled reader. The detailed construction and specification for the different structures are considered the object of a normal design procedure performed by the skilled person along the lines set out in the pre-sent specification. For example, the individual components for the disclosed embodiments may be manufactured using materials suitable for medical use and mass production, e.g. suitable polymeric materials, and assembled using cost-effective techniques such as bonding, welding, adhesives and mechanical interconnections.

1. A drug delivery device (300, 600, 700, 1000) comprising, or being adapted to be connected to, a transcutaneous access device (951, 630, 730) adapted to be arranged subcutaneously in a subject, the drug delivery device further comprising:
a reservoir (350, 620, 720) adapted to contain a fluid drug,
an expelling assembly (330, 640, 740) adapted for cooperation with the reservoir to expel fluid drug out of the reservoir and through the transcutaneous access device,
a controller (361, 650, 750) for detecting a first condition representative of the transcutaneous access device being arranged in a subcutaneous first position, and for detecting a second condition representative of the transcutaneous access device being arranged in a non-subcutaneous second position, wherein
the controller is adapted for performing an action corresponding to the detection of the second condition.

2. A drug delivery device as in claim 1, wherein the controller is adapted to:
operate the expelling assembly in accordance with a first mode,
detect a value for a property associated with operation of the expelling assembly,
provide a first value range when the expelling assembly is operated during the first mode, the first value range being indicative of a first condition of delivery of drug,
on the basis of the first value range provide a second value range, the second value range being indicative of a second condition of delivery of drug,
operate the expelling assembly in accordance with a second mode,
detect a value for the property when the expelling assembly is operated during the second mode, and
perform an action when a detected value is within the second value range.

3. A drug delivery device as in claim 2, wherein the first mode is priming of the pump and the transcutaneous access device with fluid drug, and the first condition is subcutaneous delivery of drug when the transcutaneous access device is arranged subcutaneously, and the second condition is non-subcutaneous delivery of drug.

4. A drug delivery device as in claim 1, wherein the controller is adapted to detect a first condition associated with the subcutaneous delivery of drug, and detect a second condition associated with the non-subcutaneous delivery of drug.

5. A drug delivery device as in claim 4, wherein the controller is adapted to detect a first condition associated with the pressure in the transcutaneous access device during subcutaneous delivery of drug, and to detect a second condition associated with the pressure in the transcutaneous access device during non-subcutaneous delivery of drug.

6. A drug delivery device as in claim 4, wherein the controller is adapted to detect a first condition associated with a first pressure in the transcutaneous access device during delivery of drug, and to detect a second condition associated with a lower pressure in the transcutaneous access device during delivery of drug.

7. A drug delivery device as in claim 2, wherein the controller comprises a pressure sensor (652) in fluid communication with the transcutaneous access device.

8. A drug delivery device as in claim 7, wherein the controller comprises information representing a first pressure range or pressure pattern associated with the first condition, and a second pressure range or pressure pattern associated with the second condition.

9. A drug delivery device as in claim 2, wherein the controller comprises a current sensor for sensing current supplied to the expelling assembly.

10. A drug delivery device as in claim 9, wherein the controller comprises information representing a first current range or current pattern associated with the first condition, and a second current range or current pattern associated with the second condition.

11. A drug delivery device as in claim 2, wherein the controller comprises a position sensor (528, 529, 537, 651) for sensing a position of a structure moved during operation of the expelling assembly.

12. A drug delivery device as in claim 11, wherein the expelling assembly comprises actuating means (500) moveable between first and second positions, the controller comprising detection means for detecting a lapsed time or time pattern when the actuating means is moved between the first and second positions in a given direction.

13. A drug delivery device as in claim 12, wherein the controller comprises information representing a first lapsed time range or time pattern associated with the first condition, and a second lapsed time range or time pattern associated with the second condition.

14. A drug delivery device as in claim 1, wherein the transcutaneous access device comprises or is associated with a sensor (806, 814, 825, 834, 845) influenced by a property associated with subcutaneous placement of a distal portion thereof.

15. A drug delivery device as in claim 1, further comprising a mounting surface (1020) adapted for application to a skin surface of the subject, wherein the transcutaneous access device comprises a distal end (951) adapted to be inserted through the skin of the subject, the distal end being moveable between an initial position in which the distal end is retracted relative to the mounting surface, and an extended position in which the distal end projects relative to the mounting surface.

16. A drug delivery device as in claim 1, further comprising a housing in which the reservoir and expelling assembly are at least partially arranged, the transcutaneous access device (731) being arranged outside the housing and being connected or connectable thereto by means of a flexible tube (732).

17. A drug delivery device as in claim 1, wherein the controller is adapted for actuating an alarm when a condition representative of the transcutaneous access device being arranged in a non-subcutaneous position is detected.

18. A drug delivery device as in claim 17, comprising indication means (771) adapted to indicate to a user that the transcutaneous access device is arranged in a non-subcutaneous position.

19. A drug delivery device as in claim 17, comprising a delivery unit (710) in which the reservoir and expelling assembly are arranged, and a remote unit (770) comprising indication means (771) adapted to indicate to a user that the transcutaneous access device is arranged in a non-subcutaneous position.

20. A method for operating a drug delivery device comprising a reservoir, an expelling assembly and a transcutaneous access device, comprising the steps of:
arranging the transcutaneous access device subcutaneously in a subject,
operating the expelling assembly to expel fluid drug out of the reservoir and through the transcutaneous access device,
detecting a property associated with operation of the expelling assembly,
determining a first value or range for the property with the transcutaneous access device arranged subcutaneously,
determining on the basis of the first value or range a second range indicative of the transcutaneous access device being arranged non-subcutaneously,
performing an action when a value or pattern for the property within the second range is detected during operation of the expelling assembly.

21. A method for operating a drug delivery device comprising a reservoir, an expelling assembly and a transcutaneous access device, comprising the steps of:
operating the expelling assembly to expel fluid drug out of the reservoir through the transcutaneous access device,
detecting a property associated with operation of the expelling assembly,
determining whether the detected property is indicative of drug being expelled subcutaneously in a subject or non-subcutaneously.

22. A method for operating a drug delivery device comprising a reservoir, an expelling assembly and a transcutaneous access device, comprising the steps of:
arranging the transcutaneous access device subcutaneously in a subject,
operating the expelling assembly to expel fluid drug out of the reservoir and through the transcutaneous access device,
detecting a property associated with operation of the expelling assembly,
determining whether the detected property is indicative of drug being expelled subcutaneously in a subject or non-subcutaneously.

23. A method as in claim 20, wherein the property is representative of the pressure in the transcutaneous access device during operation of the expelling assembly.

24. A method for operating a drug delivery device comprising a reservoir, an expelling assembly and a transcutaneous access device, comprising the steps of:
operating the expelling assembly to expel fluid drug out of the reservoir through the transcutaneous access device,
detecting a condition associated with the pressure in the transcutaneous access device during delivery of drug,
actuating an alarm when the detected condition is associated with a pressure below a defined value.

25. A method as in claim 20, comprising the step of indicating to a user that the transcutaneous access device is arranged in a non-subcutaneous position.

26. A method for operating a drug delivery device comprising a transcutaneous access device, comprising the steps of:
arranging the transcutaneous access device subcutaneously in a subject,
detecting a condition influenced by the transcutaneous access device being arranged in a subcutaneous or non-subcutaneous position, and
generating an alarm when a condition indicative of the transcutaneous access device being arranged in a non-subcutaneous position is detected.

27. A computer usable media including an embedded computer program that is capable of carrying out a method as in claim 19 when the computer is executed in a drug delivery device comprising a transcutaneous access device adapted to be arranged subcutaneously in a subject, a reservoir adapted to contain a fluid drug, an expelling assembly adapted for cooperation with the reservoir to expel fluid drug out of the reservoir and through the transcutaneous access device, and a controller adapted to detect a condition influenced by the subcutaneous or non-subcutaneous position of the transcutaneous access device.

28. A medical device (1000), comprising:
a transcutaneous device (951, 801, 811, 821, 831, 841, 851, 861) adapted to be arranged subcutaneously in a subject,
a controller (361, 802, 812, 822, 832, 842, 852, 862) for detecting a first condition representative of the transcutaneous device being arranged in a subcutaneous first position, and for detecting a second condition representative of the transcutaneous device being arranged in a non-subcutaneous second position, wherein
the controller is adapted for performing an action corresponding to the detection of the second condition.

29. A medical device as in claim 28, wherein the controller is adapted to:
operate the device in accordance with a first mode,
detect a value for a property associated with the first mode and provide a first value range being indicative of a first condition,
on the basis of the first value range provide a second value range, the second value range being indicative of a second condition,
operate the device in accordance with a second mode and detect a value for the property when the device is operated during the second mode, and
perform an action when a detected value is within the second value range.

30. A medical device as in claim 29, wherein the first mode is associated with the initial operation of the transcutaneous device, the first condition is associated with the transcutaneous device being arranged subcutaneously, and the second condition is associated with non-subcutaneous placement of the transcutaneous device.

31. A medical device, comprising:
a mounting surface (1020) adapted for application towards a skin surface of a subject,
a flexible transcutaneous device (871) comprising a distal portion adapted to be arranged subcutaneously in the subject through a point of insertion,
wherein the distal portion comprises a visual marking (873),
whereby the distal portion is readily identifiable by the naked eye of a user should the transcutaneous device disengage from its intended subcutaneous position.

32. A medical device as in claim 31, wherein the visual marking is in the form of a colour marking (873) arranged at the distal end of the transcutaneous device, or the visual marking is arranged along the length of the distal portion of the transcutaneous device.

33. A medical device as in claim 31, wherein, in a situation of use in which the medical device is applied towards the skin surface of the subject, the point of insertion can be observed directly by the user, this allowing the user to detect a condition in which the transcutaneous device has disengaged from its intended subcutaneous position.

34. A medical device as in claim 31, wherein the distal portion of the transcutaneous device (951) is moveable from a retracted position to an extended position relative to the mounting surface.

35. A medical device, comprising:
a mounting surface adapted for application towards a skin surface of a subject,
a first electrode (806) and a second electrode (805), and
means (802) for detecting a capacitance between the first and second electrodes,
wherein the first electrode is in the form of a transcutaneous device (801) comprising a distal portion adapted to be arranged subcutaneously in the subject, the transcutaneous device being conductive in a situation of use, and
wherein the second electrode is in the form of a skin-mountable electrode.

36. A medical device as in claim 35, wherein the transcutaneous device is conductive.

37. A medical device as in any of claims 35, wherein the transcutaneous device is hollow and substantially non-conductive, the fluid-filled transcutaneous device providing a conductive transcutaneous device in a situation of use.

38. A medical device as in claim 35, further comprising:
means (808) for applying an AC voltage between the two electrodes,
a controller for detecting a first condition determined by a first range of capacitance values representative of the transcutaneous access device being arranged in a subcutaneous first position, and for detecting a second condition determined by a second range of capacitance values representative of the transcutaneous access device being arranged in a non-subcutaneous second position, wherein
the controller is adapted for performing an action corresponding to the detection of the second condition.

39. A medical device as in claim 38, wherein the controller is adapted to:
detect a capacitance value when the medical device is operated during a first mode,
on the basis of the detected capacitance value provide a first capacitance value range, the first capacitance value range being indicative of a first condition of placement of the transcutaneous device,
on the basis of the first value range provide a second capacitance value range, the second value range being indicative of a second condition of placement of the transcutaneous device,
operate the medical device in accordance with a second mode,
detect a capacitance value when the medical device is operated during the second mode, and
perform an action when a detected capacitance value is within the second value range.

40. A medical device as in claim 39, wherein the first mode is an initial mode associated with the transcutaneous access device being arranged subcutaneously in the subject, the first condition is associated with the transcutaneous device being arranged subcutaneously with the second electrode in contact with the skin surface, and the second condition is associated with the transcutaneous device being arranged non-subcutaneously with second electrode in contact with the skin surface.

41. A medical device as in claim 35, wherein the second electrode is associated with the mounting surface.
